(19) United States
(12) Patent Application Publication
(10) Pub. No.: US 2012/0257166 A1
(43) Pub. Date: Oct. 11, 2012

(54) PORTABLE SELF-RETINAL IMAGING DEVICE

(75) Inventors: Robert Paul Francis, Lewisville, TX (US); Jack Christopher Smith, Parker, TX (US)

(73) Assignee: RAYTHEON COMPANY, Waltham, MA (US)

(21) Appl. No.: 13/440,464

(22) Filed: Apr. 5, 2012

Related U.S. Application Data
(60) Provisional application No. 61/472,986, filed on Apr. 7, 2011.
(60) Provisional application No. 61/491,502, filed on May 31, 2011.

(51) Int. Cl. A61B 3/15; A61B 3/12; A61B 3/14

(52) U.S. Cl. 351/208; 351/206; 351/246

(57) ABSTRACT

A portable MEMS-based scanning laser ophthalmoscope (MSLO). In one example, the MSLO includes a laser illumination sub-assembly, a two-dimensional MEMS scanning mirror, a conic front objective, and a detector sub-assembly all disposed within a portable housing. A battery configured to provide power to components of the MSLO may also be included within the housing. In one example, the laser illumination sub-assembly includes at least one laser configured to generate in each of two orthogonal dimensions one or more illumination beams separated from one another by a predetermined angle of separation. The MEMS scanning minor and conic front objective are configured to produce a two-dimensional area of illumination from the illumination beam(s) in each dimension and to direct the illumination from the scanning minor to the eye to illuminate the retina.

### CROSS REFERENCE TO RELATED APPLICATIONS

This application claims the benefit under 35 U.S.C. §119(e) of co-pending U.S. Provisional Patent Application No. 61/472,986 titled “PORTABLE SELF-RETINAL IMAGING DEVICE” filed on Apr. 7, 2011 and of co-pending U.S. Provisional Patent Application No. 61/491,502 titled “PORTABLE SELF-RETINAL IMAGING DEVICE” filed on May 31, 2011, both of which are incorporated herein by reference in their entireties.

### BACKGROUND

Ophthalmic fundus cameras have been used by ophthalmic specialists for many years to image the interior surface of the eye (the retina), including the fundus, optic disc, macula and fovea, and posterior pole. Generally, a fundus camera has approximately a 30 to 45 degree spherical field of view on the retina. These cameras operate on the principle of direct or indirect ophthalmoscopy, and flood the eye with light from a flash bulb and capture a two-dimensional image with imaging optics and a detector. The light from the flash bulb is focused via a series of lenses through a doughnut-shaped aperture, and then passes through a central aperture to form an annulus before passing through the camera objective lens and through the cornea onto the retina. The light reflected from the retina passes through the un-illuminated hole in the doughnut formed by the illumination system to a telescopic eyepiece. To obtain an image of the retina, a mirror interrupts the path of the illumination system to allow the light from the flash bulb to pass into the eye, and simultaneously, a minor falls in front of the observation telescope to redirect the light onto the detector. These instruments are complex in design and difficult to manufacture to clinical standards. In addition, fundus cameras are limited to a relatively small field of view and worse than diffraction-limited resolution on the retina due to aberrations introduced by the imaging optics and the front objective common to both illumination and imaging paths. Portable or handheld fundus cameras are commercially available, but are not widely used because they require a skilled photographer for operation and the images captured are poor relative to tabletop devices.

Another device used to obtain images of the retina is the scanning laser ophthalmoscope, which is generally able to image the retina with better spatial resolution than a fundus camera. The scanning laser ophthalmoscope uses a laser illuminator which is raster scanned over the retina and a detector configured to measure light reflected from the retina at each point in the scan. The scanning elements used to scan the illuminator include rotating polygons, scanning prisms and galvanometer-driven movable minors. These elements are difficult to align and sensitive to shock and vibration, making their use impractical in portable systems.

For wide field of view (FOV) imaging, existing systems use an elliptical minor for virtual point scanning in the retina. The real scan point is placed on one focus of the ellipse and the other focus of the ellipse is located in the pupil of the human eye. If the scan is symmetric about the minor axis of the ellipse, then the virtual scan angle is equal to the real scan angle.

### SUMMARY OF INVENTION

Aspects and embodiments are directed to a scanning laser ophthalmoscope that replaces conventional scanning elements with a two-dimensional MEMS (microelectromechanical systems) scanning minor, thereby enabling robust scanning in a portable device, as discussed further below. The MEMS-based scanning laser ophthalmoscope may be small in size and weight and can be operated on battery power, allowing for a person-portable device which may be operated in remote locations. Embodiments of the ophthalmoscope may be configured with continuous and real-time feedback for alignment and focus, as discussed further below.

According to one embodiment, a MEMS-based scanning laser ophthalmoscope comprises a laser illumination sub-assembly configured to generate one or more laser illumination beams, a two-dimensional MEMS scanning mirror configured to receive the laser illumination beam(s) and to produce a two-dimensional area of illumination, an optical system optically coupled to the MEMS scanning minor and configured to direct the two-dimensional area of illumination from the scanning minor into an eye to illuminate a retina of the eye, and a detector sub-assembly optically coupled to the optical system and the MEMS scanning mirror and configured to intercept optical radiation reflected from the eye to generate an image of the retina.

In one example of the MEMS-based scanning laser ophthalmoscope, the detector sub-assembly includes a photodetector and a holed minor, the holed mirror being positioned over the two-dimensional MEMS scanning minor and configured and arranged to allow the laser illumination beams to pass through an opening in the holed mirror to the optical system, and to direct the optical radiation reflected from the eye to the photodetector. The photodetector may include, for example, an avalanche photodiode, a charge coupled device, or a photo-multiplier tube. In one example, the detector sub-assembly further includes a focusing optic optically coupled to the holed mirror and configured to focus the optical radiation to the photodetector. The detector sub-assembly may further include a confocal aperture optically coupled between the focusing optic and the photodetector. In another example, the one or more laser illumination beams include one or more first illumination beams spaced apart from one another by a first angle of separation in a first dimension, and one or more second illumination beams spaced apart from another by a second angle of separation in a second orthogonal dimension. The first angle of separation may be approximately equal to the second angle of separation. In one example, the optical system includes a conic front objective. The conic front objective may have two foci, wherein the two-dimensional MEMS scanning mirror is located at a first focus of the conic front objective and the MEMS-based scanning laser ophthalmoscope is configured to accommodate a pupil of the eye at a second focus of the conic front objective. The conic front objective may be, for example, an ellipsoid objective including an ellipsoidal minor, or a double paraboloid objective. The laser illumination sub-assembly may include a near-infrared laser source and/or at least one visible laser source. In one example, the MEMS-based scanning laser ophthalmoscope further comprises a battery configured to provide power to the two-dimensional MEMS scanning minor and to the laser illumination sub-assembly. The battery may be configured to, or coupled to circuitry configured to use the power supplied by the battery to, provide a variable voltage to the two-dimensional MEMS scanning mirror to actuate the two-dimensional MEMS scanning mirror to move over a range of angular deflection in each of a first dimension and a second dimension. In another example, the MEMS-based scanning laser ophthalmoscope further comprises a display screen optically coupled to the optical system, and a controller configured to control the laser illumination sub-assembly to display a fixation target on the display screen. The controller may be further configured to adjust a display location of the fixation target on the display screen to guide an orientation of the eye so as to obtain an image of a selected region of the retina.

According to another embodiment, a portable MEMS-based scanning laser ophthalmoscope for scanning a retina of an eye comprises a housing, a laser illumination sub-assembly disposed within the housing and including at least one laser and configured to generate in each of two orthogonal dimensions one or more illumination beams separated from one another by a predetermined angle of separation, and a two-dimensional MEMS scanning minor disposed within the housing and optically coupled to the a laser illumination sub-assembly and configured to produce a two-dimensional area of illumination from the illumination beam(s) in each dimension. The portable MEMS-based scanning laser ophthalmoscope further comprises a conic front objective disposed within the housing and optically coupled to the MEMS scanning minor and configured to direct the illumination from the scanning mirror to the eye to illuminate the retina, a detector sub-assembly disposed within the housing and optically coupled to the conic front objective and the MEMS scanning minor and configured to intercept optical radiation reflected from the eye to generate an image of the retina, and a battery disposed within the housing and configured to provide power to the scanning mirror and to the laser illumination sub-assembly.

In one example of the portable MEMS-based scanning laser ophthalmoscope, the detector sub-assembly includes a photodetector and a holed minor, the holed mirror being positioned over the two-dimensional MEMS scanning minor and configured and arranged to allow the illumination beams to pass through an opening in the holed mirror to the conic front objective, and to direct the optical radiation reflected from the eye to the photodetector. The photodetector may include, for example, an avalanche photodiode, a charge coupled device, or a photo-multiplier tube. In one example, the predetermined angle of separation between the one or more illumination beams in a first dimension is approximately equal to the predetermined angle of separation between the one or more illumination beams in a second dimension. In another example, the conic front objective has two foci, and wherein the two-dimensional MEMS scanning mirror is located at a first focus of the conic front objective and the MEMS-based scanning laser ophthalmoscope is configured to accommodate a pupil of the eye at a second focus of the conic front objective. The conic front objective may be, for example, an ellipsoid objective including an ellipsoidal mirror, or a double paraboloid objective. The laser illumination sub-assembly may include at least one of a near-infrared laser source and a visible laser source. In one example, the battery is configured to provide a variable voltage to the two-dimensional MEMS scanning minor to actuate the two-dimensional MEMS scanning mirror to move over a range of angular deflection in each of the two orthogonal dimensions. The MEMS-based scanning laser ophthalmoscope may further comprise a display screen disposed within the housing and optically coupled to the conic front objective, and a controller disposed within the housing and configured to control the laser illumination sub-assembly to display a fixation target on the display screen. In one example, the controller is further configured to adjust a display location of the fixation target on the display screen to guide an orientation of the eye so as to obtain an image of a selected region of the retina of the eye. The laser illumination sub-assembly may include a visible laser source configured to provide visible laser illumination, wherein the visual laser illumination is modulated to produce the fixation target displayed on the display screen.

Another embodiment is directed to a method of imaging a retina of an eye with a scanning laser ophthalmoscope, the method comprising acts of generating laser illumination, scanning the laser illumination about a scan point at the eye using a two-dimensional MEMS scanning minor to produce a two-dimensional area of illumination that illuminates the retina of the eye, intercepting optical radiation reflected from the eye, acquiring a first image of the eye from the optical radiation, analyzing the first image of the eye to identify features in the first image, and based on the features, automatically adjusting at least one of an alignment and a focus of optical components of the scanning laser ophthalmoscope to obtained a focused image of a selected region of the retina of the eye.

In one example of the method, analyzing the image of the eye includes determining whether a pupil of the eye is centered with respect to the laser illumination, and the method further comprises laterally moving optical components of the scanning laser ophthalmoscope and acquiring subsequent images of the eye until the pupil is centered in one of the subsequent images. In another example, analyzing the image of the eye includes determining whether the retina of the eye is in focus, and the method further comprises moving the optical components of the scanning laser ophthalmoscope and acquiring additional images of the eye until the retina is in focus in one of the additional images. The method may further comprise displaying a fixation target to guide an orientation of the eye so as to obtain an image the selected region of the retina of the eye. In one example, the method further comprises adjusting a display location of the fixation target to guide the orientation of the eye so as to obtain an image of another selected region of the retina of the eye. The method may also comprise an act of providing an audio instruction to direct a patient to look at the fixation target. In one example, generating the laser illumination includes generating in each of two orthogonal dimensions one or more illumination beams separated from one another by a predetermined angle of separation.

According to another embodiment, a method of imaging a retina of an eye with a scanning laser ophthalmoscope comprises acts of generating one or more first illumination beams separated from one another in a first dimension by a first angle of separation, generating one or more second illumination beams separated from one another in a second dimension by a second angle of separation, the second dimension being orthogonal to the first dimension, scanning the one or more first and one or more second illumination beams about a scan point at the eye using a two-dimensional MEMS scanning minor to produce a two-dimensional area of illumination that illuminates the retina of the eye, intercepting optical radiation reflected from the eye, and producing an image of the retina from the optical radiation.

In one example of the method, generating the one or more first and one or more second illumination beams includes generating infra-red illumination beams. In another example, generating the one or more first and one or more second illumination beams includes generating visible illumination beams. In one example, intercepting the optical radiation reflected from the eye includes detecting the optical radiation using one of a photo-multiplier tube, a charge coupled device, and an avalanche photodiode. In another example, scanning the one or more first and one or more second illumination beams includes applying a variable voltage to the two-dimensional MEMS scanning mirror to actuate the two-dimensional MEMS scanning mirror to move over a range of angular deflection in each of the first and second dimensions.

Still other aspects, embodiments, and advantages of these exemplary aspects and embodiments, are discussed in detail below. Any embodiment disclosed herein may be combined with any other embodiment in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. In the figures, each identical or nearly identical component that is illustrated in various figures is represented by a like numeral. For purposes of clarity, not every component may be labeled in every figure. In the figures:

FIG. 1A is a functional block diagram of one example of a MEMS-based scanning laser ophthalmoscope according to aspects of the invention;

FIG. 1B is a schematic diagram illustrating one example of a configuration of a MEMS-based scanning laser ophthalmoscope according to aspects of the invention;

FIG. 2A is a graph illustrating the relationship between the angle of reflection from a parabolic reflector and the distance of the reflection from the axis of the paraboloid;

FIG. 2B is a three-dimensional graph illustrating an example of the relationship between angular magnification and focal length for a double paraboloid front objective;

FIG. 3A is a diagram illustrating one example of beam magnification/demagnification with a double paraboloid front objective;

FIG. 3B is a diagram illustrating one example of beam magnification/demagnification with an ellipsoid front objective;

FIG. 4 is a diagram of one example of a MEMS-based scanning laser ophthalmoscope configuration according to aspects of the invention;

FIG. 5A is a diagram illustrating reflection of two incident optical beams from a scanning mirror is a first position, according to aspects of the invention;

FIG. 5B is a diagram illustrating reflection of the two incident optical beams from the scanning mirror in a second position, according to aspects of the invention;

FIG. 5C is a diagram illustrating reflection of the two incident optical beams from the scanning mirror in a third position, according to aspects of the invention;

FIG. 5D is a diagram corresponding to an overlay of FIGS. 5A-5C;

FIG. 6 is a non-sequential ray trace of an example of an ellipsoid reflector with a scanning mirror positioned at two different points within its range of movement at two time points, according to aspects of the invention;

FIG. 7A is a diagram illustrating four illumination beams reflected from a scanning minor in a first position, according to aspects of the invention;

FIG. 7B is a diagram illustrating the four illumination beams reflected from the scanning mirror in a second position, according to aspects of the invention;

FIG. 7C is a diagram illustrating the four illumination beams reflected from the scanning mirror in a third position, according to aspects of the invention;

FIG. 8 is a functional block diagram of one example of a laser illumination sub-assembly according to aspects of the invention;

FIG. 9 is a schematic diagram of one example configuration of a MEMS-based scanning laser ophthalmoscope according to aspects of the invention;

FIG. 10 is a functional block diagram of one example of a detector sub-assembly for a MEMS-based scanning laser ophthalmoscope according to aspects of the invention;

FIG. 11 is a schematic diagram of another example configuration of a MEMS-based scanning laser ophthalmoscope according to aspects of the invention;

FIG. 12 is a schematic diagram of another example configuration of a MEMS-based scanning laser ophthalmoscope according to aspects of the invention;

FIG. 13 is a schematic diagram of another example configuration of a MEMS-based scanning laser ophthalmoscope according to aspects of the invention;

FIG. 14 is a schematic diagram of another example configuration of a MEMS-based scanning laser ophthalmoscope according to aspects of the invention;

FIG. 15 is a schematic diagram of another example configuration of a MEMS-based scanning laser ophthalmoscope according to aspects of the invention;

FIG. 16 is a schematic diagram of one example of a portable MEMS-based scanning laser ophthalmoscope in a housing, according to aspects of the invention;

FIG. 17 is a schematic diagram of another example configuration of a MEMS-based scanning laser ophthalmoscope according to aspects of the invention;

FIG. 18 is a schematic diagram of another example configuration of a MEMS-based scanning laser ophthalmoscope including a display screen according to aspects of the invention;

FIGS. 19A-C are a flow diagram of one example of a retinal imaging process according to aspects of the invention;

FIG. 20A is an example of an image of a display screen presented to a patient using an embodiment of the MEMS-based scanning laser ophthalmoscope, according to aspects of the invention;

FIG. 20B is another example of an image of the display screen according to aspects of the invention;

FIG. 20C is another example of an image of the display screen according to aspects of the invention;

FIG. 20D is another example of an image of the display screen according to aspects of the invention;

FIG. 21A is a schematic diagram illustrating a first position of a patient's eye with respect to the illumination beam of a MEMS-based scanning laser ophthalmoscope according to aspects of the invention;

FIG. 21B a schematic diagram illustrating a second position of the patient's eye with respect to the illumination beam of the MEMS-based scanning laser ophthalmoscope according to aspects of the invention;

FIG. 21C is a schematic diagram illustrating a third position of the patient's eye with respect to the illumination beam of the MEMS-based scanning laser ophthalmoscope according to aspects of the invention;

FIG. 21D is a schematic diagram illustrating a fourth position of the patient's eye with respect to the illumination beam of the MEMS-based scanning laser ophthalmoscope according to aspects of the invention;

FIG. 22A an image of an eye corresponding to the eye position of FIG. 21A;

FIG. 22B is an image of the eye corresponding to the eye position of FIG. 21B;

FIG. 22C is an image of the eye corresponding to the eye position of FIG. 21C; and

FIG. 22D is an image of the eye corresponding to the eye position of FIG. 21D.

### DETAILED DESCRIPTION

Aspects and embodiments are directed to a compact, wide-field scanning laser ophthalmoscope configured to enable handheld, portable use, for example, in remote locations and primary-care-physician offices, and for self-administered retinal imaging. Portable, self-administered retinal imaging would be invaluable for screening remote populations for eye disease, and for screening warfighters for ocular injury in the battlefield, to monitor immediate ocular effects of battlefield trauma. Similarly, retinal imaging in a physician's office would greatly improve the efficiency of screening diabetics for retinopathy, for example. Conventional table-top retinal imaging devices are too large for such applications and/or require a trained expert to operate.

According to one embodiment, self-administered, wide-field imaging of the retina in a compact, portable hardware footprint is achieved with a MEMS-based scanning laser ophthalmoscope (MSLO). To enable robust scanning in a portable device, a two-dimensional (2D) MEMS scanning mirror replaces conventional scanning elements, such as the rotating polygons, scanning prisms and galvanometer-driven movable minors discussed above. In addition, an optical system, for example, a conic front objective, is used to magnify the scan angle to allow for scanning over approximately a 100 degree or greater spherical field of view on the retina. As discussed further below, embodiments of the MSLO use multiple light paths/angles to multiply the effective scan range, and a holed mirror surrounding the scan mirror to collect scattered light and return more light to the detector. In addition, embodiments of the MSLO are configured to present fixation targets to human subjects with real-time feedback to enable fully automated, self-administered retinal imaging, as also discussed further below.

Embodiments of the MSLO may enable retinal imaging outside of the traditional ophthalmologist office, including applications such as, for example, diabetic retinopathy screening using a telemedicine network, military ocular injury imaging in the field, retinal imaging in under-served locations of the world, and home care providers using portable systems. Another advantage of the MSLO is that it can be operated with low-light, so no pupil dilation is required.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiment. Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

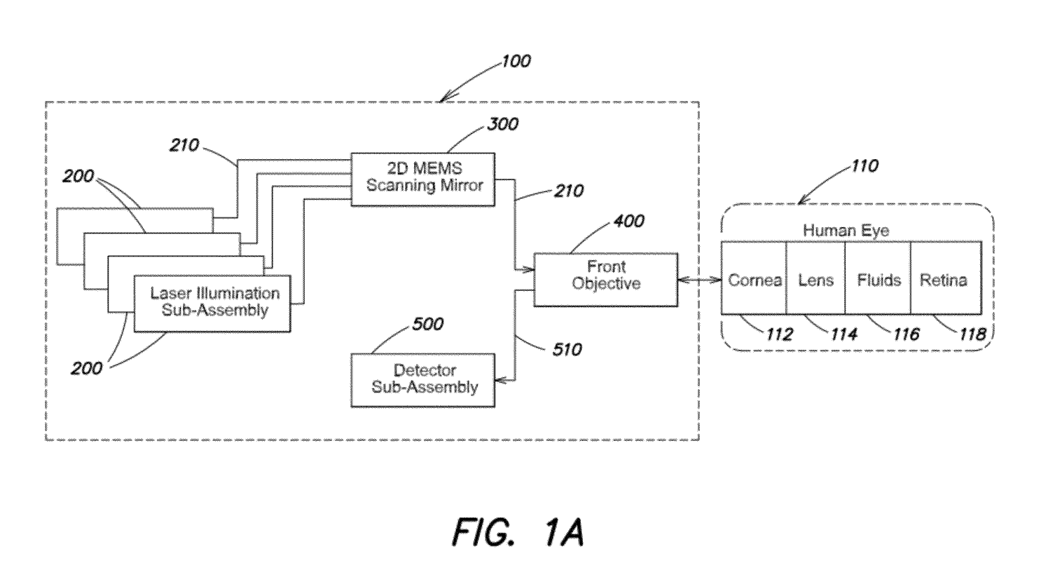

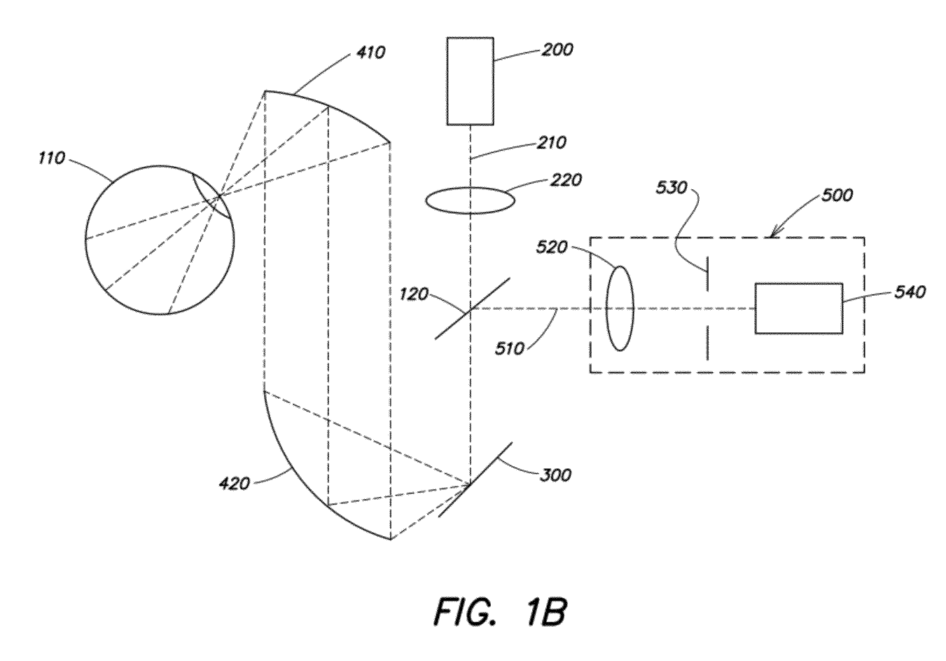

Referring to FIG. 1A there is illustrated a functional block diagram of one example of an MSLO according to one embodiment. FIG. 1B illustrates an example of a corresponding configuration of the MSLO. As discussed in more detail below, the MSLO 100 may include one or more laser illumination sub-assemblies 200 that generate optical illumination beams 210 for scanning the human eye 110. The laser illumination sub-assembly 200 may include or may be coupled to focusing optics 220 to focus and/or collimate the illumination beams 210. The illumination beams 210 are scanned angularly in two dimensions about a virtual scan point (VSP) in the pupil of the eye 110. This two-dimensional scan is achieved using the 2D MEMS scanning minor 300 and a conic front objective 400 having two focal points, such that a scanning element can be placed at one focal point, referred to as the real scan point (RSP), and the pupil of the eye 110 can be placed at the other focal point, the VSP.

In one example, the conic front objective 400 is a double paraboloid, as illustrated in FIG. 1B, including two minors 410, 420 each of which has a sectional paraboloid shape (i.e., the surface of the mirror has a shape corresponding to a section of a three-dimensional parabola). In another example, the conic front objective is ellipsoid, comprising a minor having a surface shape corresponding to section of an ellipse. Embodiments of the conic front objective 400 are discussed further below. The illumination beams 210 travel through the cornea 112, the lens 114 and fluids 116 and are incident on the retina 118. Light scattered by the retina 118 travels back through the eye and is directed by the conic front objective 400 into a return beam 510 that is detected by an optical detector sub-assembly 500, as discussed further below. In one example, the detector sub-assembly 500 includes focusing optics 520 and a confocal aperture 530 to direct and focus the return beam 510 onto a detector 540, as discussed further below. A beam splitter 120 may be used to appropriately direct the illumination beams 210 and return beam 510 and allow these beams to share a common optical pathway.

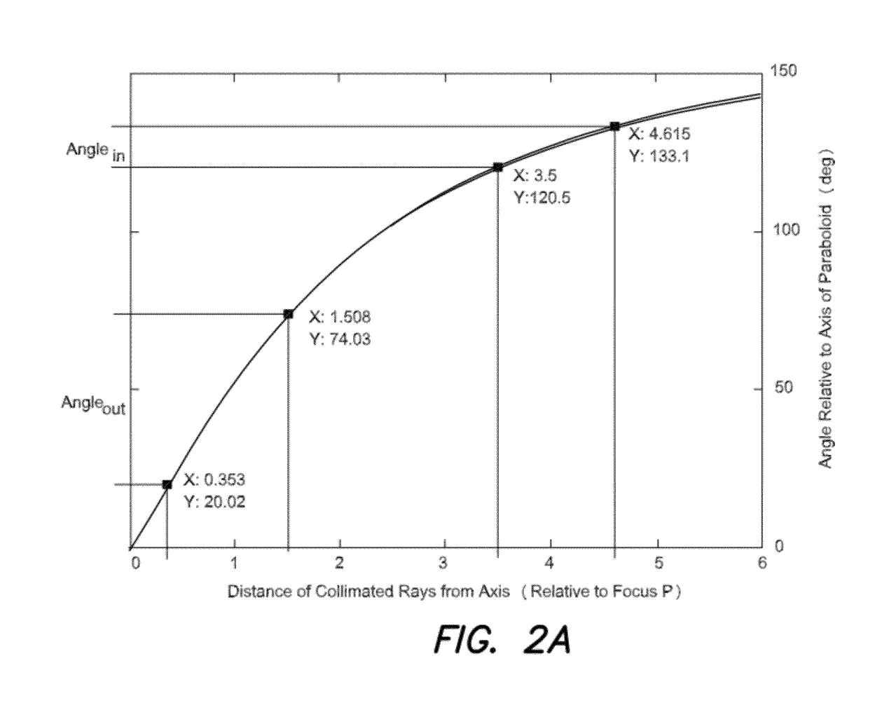

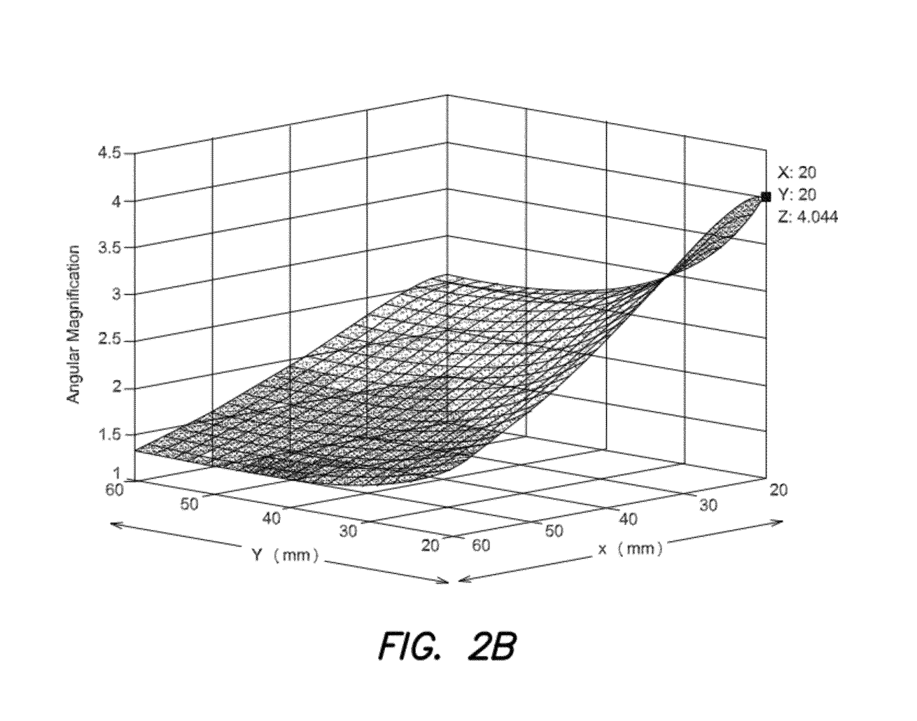

The relationship between the angle of deflection of the illumination beams 210 at the RSP (the scanning minor 300), called the real scan angle (RSA), and the angle of deflection at the VSP, called the virtual scan angle (VSA), is determined by the curvature and change of curvature in the surface of the front objective 400 over the spatial extent of the scan. As illustrated in FIG. 2A, the angle of reflection from a parabolic reflector is a function of the distance of the reflection from the axis of the paraboloid. If two paraboloids with parallel axes are facing each other, the VSA at the focus of one paraboloid can be adjusted with respect to the RSA at the focus of the other paraboloid by offsetting the axes of the two paraboloids so that the reflection points are different distances from the axes in each paraboloid. Similarly, if the front objective 400 is an ellipsoid, different segments of the ellipsoid will result in angular magnification or demagnification, meaning that the ratio of VSA to RSA can be less than, equal to, or greater than 1.

For example, referring to FIGS. 2A and 2B, if the parabolic focus of the first paraboloid is the same as the parabolic focus of the second paraboloid, then the angle magnification is given by the ratio of the angle (relative to the axis of the paraboloid) of the reflected beam and the angle (relative to the axis of the paraboloid) of the incident beam. In the example illustrated in FIG. 2A, the angle magnification is (74.03−20.02)/(133.1−120.5)=55.01/12.6=4.3. FIG. 2B illustrates that the angular magnification of the double paraboloid front objective is also a function of the focal lengths of the two paraboloids, with shorter focal lengths providing greater angular magnification. In FIG. 2B, the focal lengths of the two paraboloids (in mm) are represented on the x and y axes, and angular magnification is represented on the z axis.

To illuminate a diffraction-limited spot on the retina, the beam entering the eye 110 must be approximately 1-2 millimeters (mm; 0.039-0.079 inches) in diameter, and nearly collimated. This beam diameter is determined by the optical properties of the human eye, with a 1 mm beam providing approximately the smallest/finest resolution in the eye. In one example, a 1 mm diameter beam at the cornea 112 enables a ten micron (0.0004 inches) spot size on the retina. As a result of this desired beam diameter, the beam at the pupil includes not only rays through the VSP, but also those at a distance of half the beam diameter from the VSP. These rays, therefore, will not travel through the RSP, which results in a beam diameter magnification or demagnification inversely proportional to the angular magnification or demagnification discussed above.

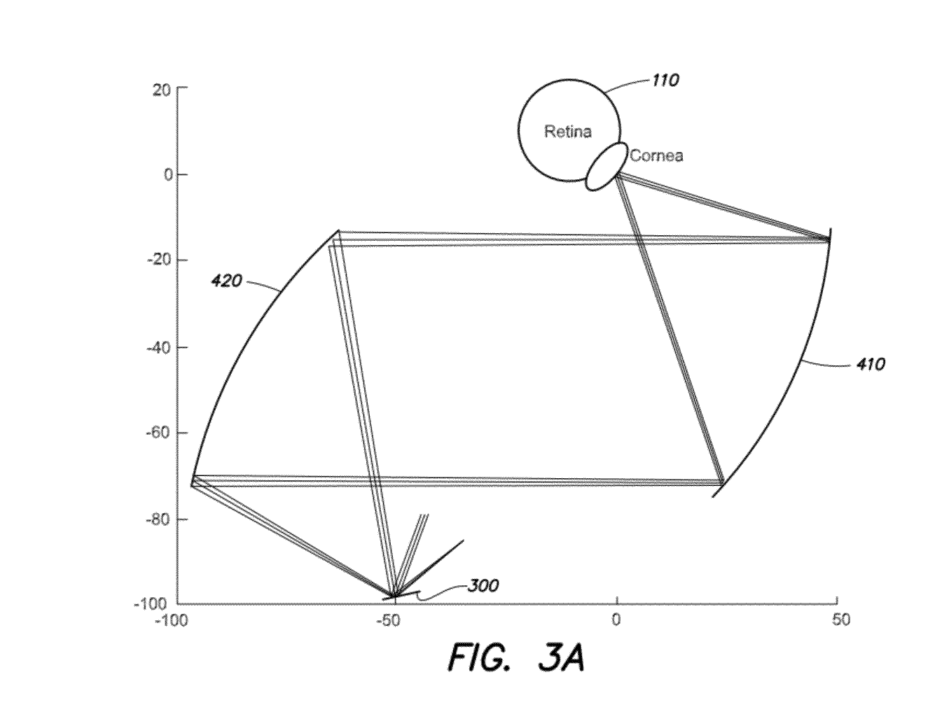

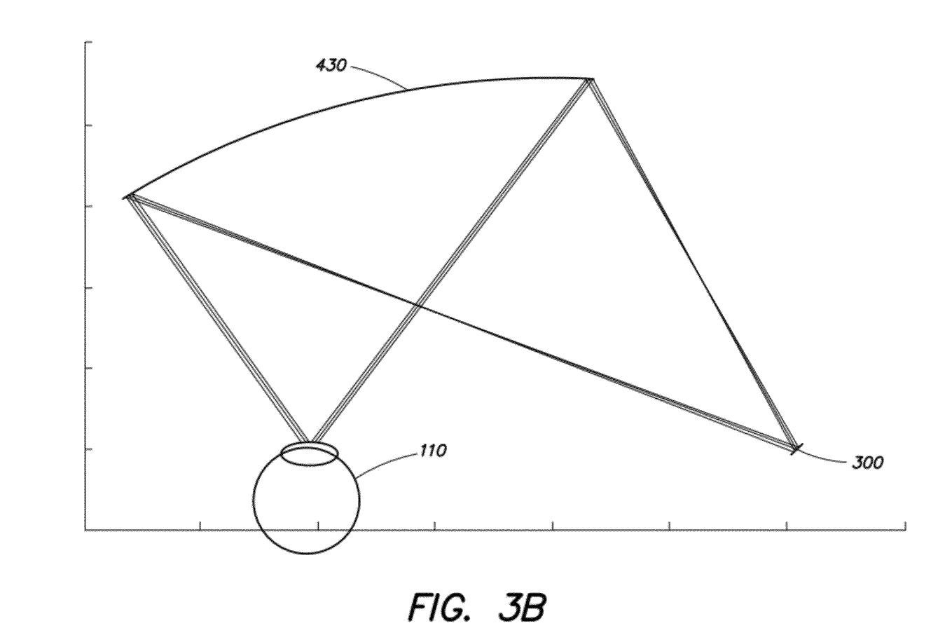

FIG. 3A illustrates an example of beam magnification/demagnification with a double paraboloid front objective 400 including a first paraboloid 410 and a second paraboloid 420. As discussed above, the scan minor 300 is placed at the RSP to direct the illumination beams to the eye 110, the pupil of which is at the VSP. FIG. 3B illustrates a similar example of beam magnification/demagnification with the front objective 400 comprising an ellipsoid 430.

According to one embodiment, it is desirable to have a wide-field MSLO configured to accomplish scanning over approximately a 100 degree or greater spherical field of view on the retina 118, with the scanning beam having a beam diameter at the VSP (pupil of the eye) of approximately 1 mm. In one example, the 2D MEMS scanning mirror 300 has a resonant frequency of greater than 1 kilohertz (kHz), is approximately 2 mm in diameter and has a mechanical deflection angle maximum of approximately 10-12 degrees peak-to-peak. In this example, the conic front objective 400 is configured to magnify the scan angle to achieve a VSA of approximately 54 degrees or greater, so as to obtain the desired 100 degree or greater spherical field of view on the retina 118. As discussed above, scan angle magnification from a curved front objective results in beam diameter demagnification. Accordingly, in one example, to scan a 1 mm diameter beam with a VSA of 54 degrees or greater, while limiting the beam diameter at the RSP to less than 2 mm (the diameter of the example MEMS scanning mirror 300), requires an RSA of greater than 40 degrees.

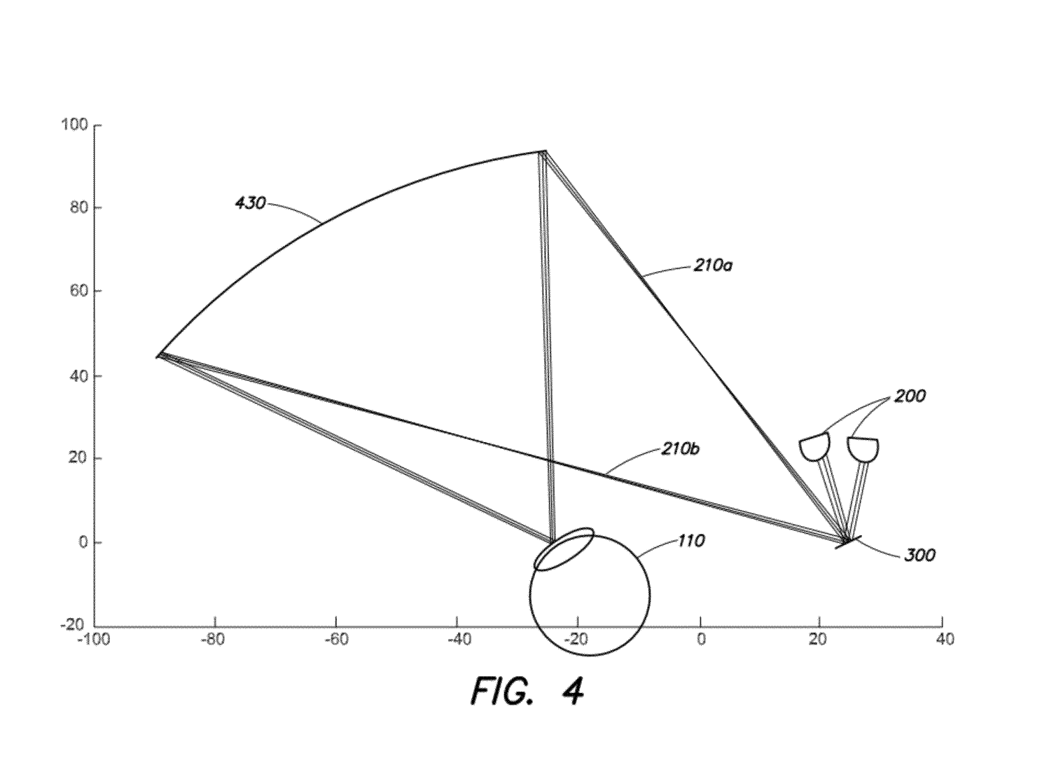

According to one embodiment, in order to solve the paradox of scan angle magnification versus beam diameter demagnification, multiple light paths of different angles are incident on the same scanning minor 300, as illustrated in FIG. 4. In the illustrated example, two laser illumination sub-assemblies 200 each generate an illumination beam 210a, 210b, respectively, with the two illumination beams being incident on the scanning mirror 300 and a predetermined angle of separation between the beams. Each illumination beam 210a, 210b has a different scan angle based on the different segments of the ellipse 430 used to reflect the beams. Similarly, if the front objective were a double paraboloid (as illustrated in FIG. 3A, for example) instead of an ellipse, the scan angles for the different beams would differ based on the different segments of the paraboloids used to reflect the beams. The combination of the scan angles for the multiple beams results in a larger (or magnified) overall scan angle, without a corresponding demagnification of the beam diameter. Thus, for the example discussed above, a 40 degree RSA may be achieved using the two laser illumination sub-assemblies 200 with a 20 degree angle of separation combined with the 10 degrees of mechanical scan range provided by the example of the scanning mirror 300.

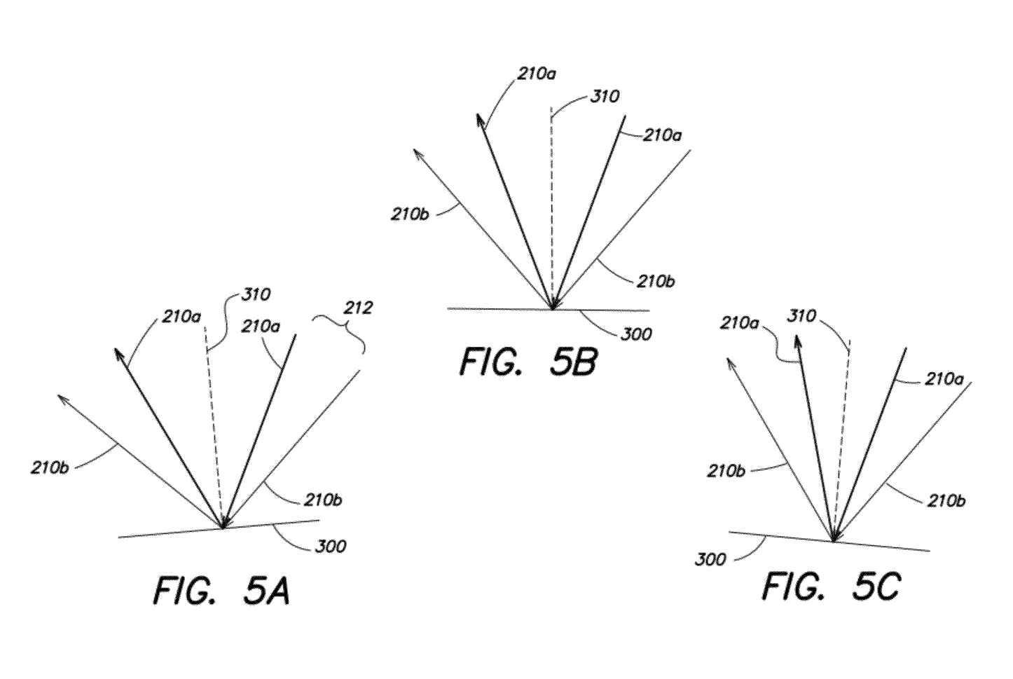

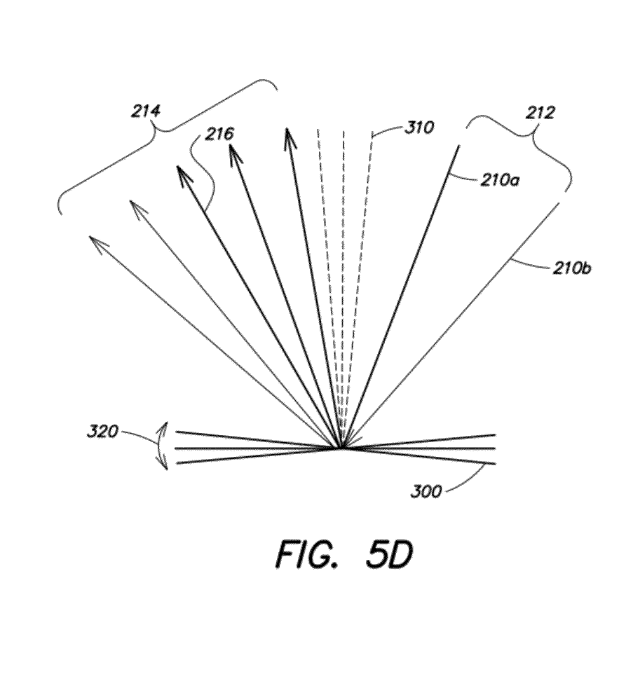

FIGS. 5A-5D provide an illustration of expanding the scan angle using mechanical movement of the MEMS scanning mirror 300 and multiple illumination beams. Referring to FIGS. 5A-5C, two illumination beams 210a and 210b are incident on the scanning minor 300 and reflected from the scanning minor. There is an angle of separation 212 between the two beams 210a, 210b. FIG. 5A illustrates the scanning minor 300 rotated to a maximum angle in one direction, FIG. 5B illustrates the scanning mirror in its neutral or mid-point position, and FIG. 5C illustrates the scanning mirror rotated to a maximum angle in the opposite direction from that illustrated in FIG. 5A. FIGS. 5A-5C illustrate rotation of the scanning mirror 300, and subsequent movement in the reflected beams 210a, 210b in one dimension. It will be appreciated by those skilled in the art, given the benefit of this disclosure, that for two-dimensional scanning, the scanning mirror may be similarly moved in a second, orthogonal dimension, as discussed further below. Line 310 represents the normal to the surface of the scanning minor 300. FIG. 5D is an overlay of the diagrams of FIGS. 5A-5C. As can be seen with reference to FIG. 5D, the angle of separation 212 between the two illumination beams 210a, 210b together with the range of movement 320 of the scanning minor 300 produce an increased overall scan angle 214 for the illumination beams reflected from the scanning mirror. In FIG. 5D, ray 216 represents both overlapped reflected beams 210a and 210b. For the example discussed above, an angle of separation 212 of 20 degrees coupled with a mechanical range of movement 320 of approximately 10 degrees can produce a scan angle 214 of approximately 40 degrees. However, it is to be appreciated that numerous variations may be implemented to achieve numerous different desired scan angles 214. For example, two or more illumination beams 210 may be used with any of numerous angles of separation between them. In another example, the range of movement of the scanning mirror 300 may be more or less than 10 degrees peak-to-peak. In addition, the angles of incidence of the two or more illumination beams 210 on the scanning minor may be selected to utilize desired segments of the front objective 400.

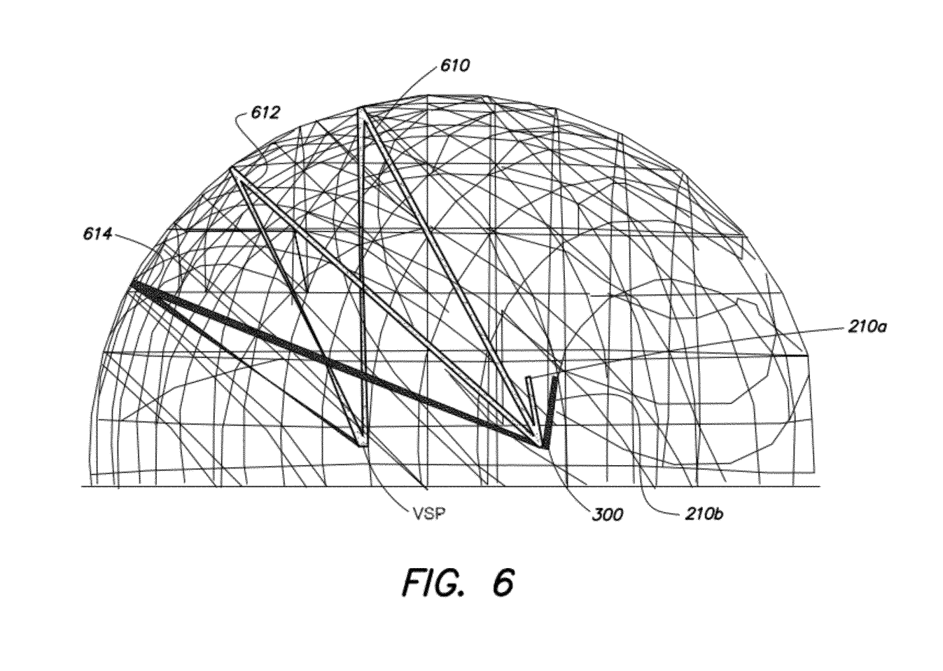

FIG. 6 illustrates a non-sequential ray trace of an example of an ellipsoid reflector with the scan minor 300 positioned at two different points within its range of movement 320 at two time points. In the example illustrated in FIG. 6, there are two illumination beams 210a, 210b. At the first time point, with the scan minor 300 in a first position, the first incident illumination beam 210a generates a first reflected illumination beam 610, and the second incident illumination beam 210b generates a second reflected illumination beam 612. At the second time point, with the scan mirror in a second position, the first incident illumination beam 210a generates a third reflected illumination beam that is overlapped with the second reflected illumination beam 612, and the second incident illumination beam 210b generates a fourth reflected illumination beam 614.

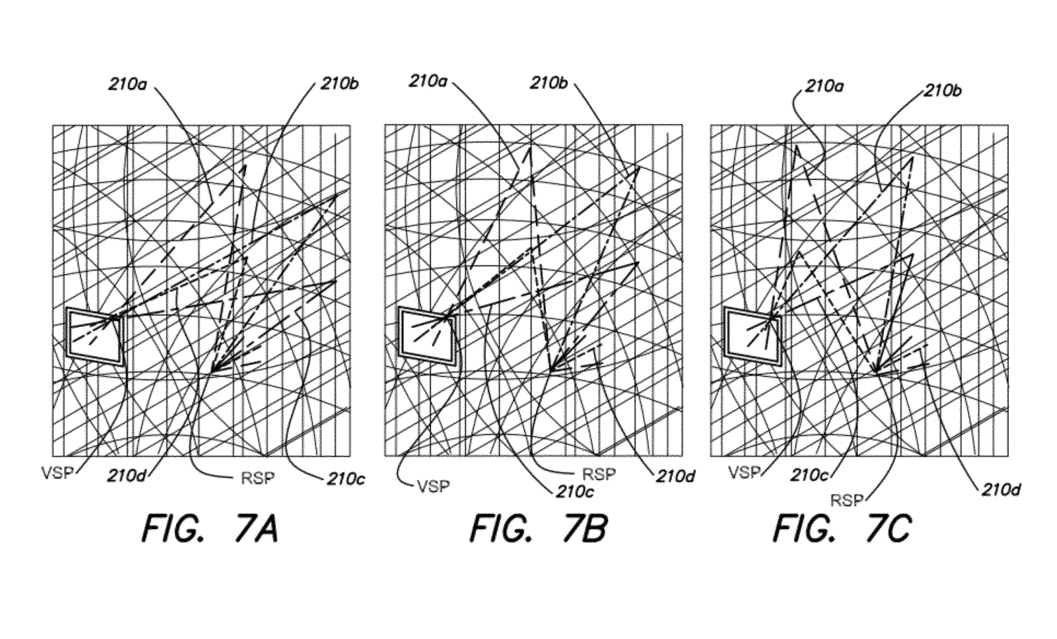

FIG. 6 illustrates the ray-trace for one dimension of the scan. For a similar example of a two-dimensional scan, there are four incident light paths or illumination beams 210a, 210b, 210c and 210d separated by a selected angle of separation in the x-dimension and separated by another selected angle of separation in the y-dimension, as illustrated in FIGS. 7A-7C. FIGS. 7A-7C illustrate different time points corresponding to different positions of the scanning minor 300 which is placed at the RSP. In the illustrated example, the illumination beams are separated by 20 degrees in x-dimension and 20 degrees in the y-dimension, and FIG. 7A represents the scanning mirror positioned at +5 degrees deflection, FIG. 7B represents the scanning mirror positioned at 0 degrees deflection, and FIG. 7C represents the scanning mirror position at −5 degrees deflection.

According to one embodiment, by modulating each of the four lasers generating the illumination beams 210a-d at a rate much faster than the scan rate of the mirror 300, the time to scan a wide field of regard can be reduced. For example, if the field of regard is 2000 by 2000 individual pixel measurements, and the maximum scan rate of the minor 300 in one dimension is 1 kHz, with one incident laser, two seconds are required for a full scan and 2000 individual detections must be acquired within each line scan. By contrast, with four illumination beams 210a-d, each one covering a 1000 by 1000 pixel area, the corresponding lasers can be modulated at 4 MHz with quarter time offset to complete a full scan in one second with only 1000 individual detections per illumination beam in each line scan. In another example, if a scanning minor 300 with less RSA mechanical deflection is used, more than two incident light paths or angles for illumination may be used in each of the x and y-dimensions; however, the total number of incident paths increase by the square of the number of angles used, and therefore the increased detector complexity resulting from an increased number of illumination angles may be considered in selecting a configuration for the MSLO.

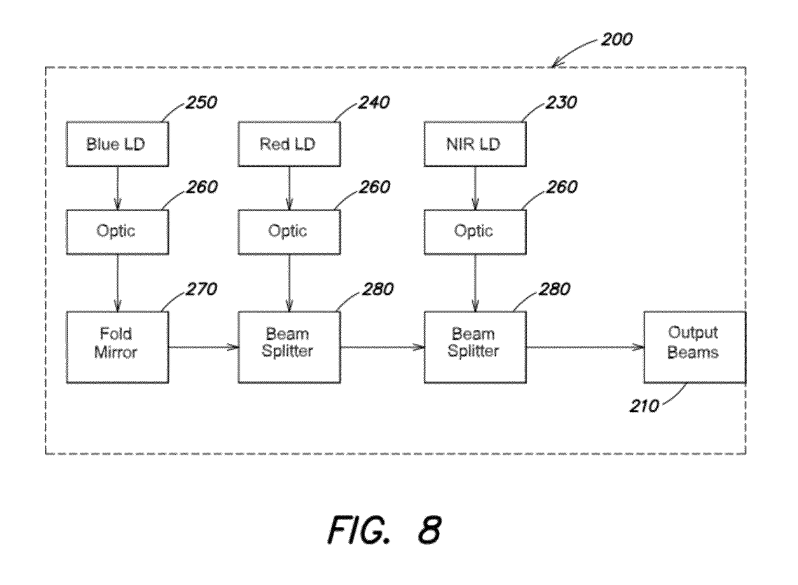

Referring to FIG. 8 there is illustrated a functional block diagram of one example of a laser illumination sub-assembly 200 according to one embodiment. The laser illumination sub-assembly 200 includes one or more lasers configured to generate the illumination beam(s) 210 at selected wavelengths. In one example, the MSLO may be configured for continuous near-infrared retinal image acquisition and image processing, and accordingly in this example the laser illumination sub-assembly includes a near-infrared laser 230. Additional wavelengths may be useful for acquiring further information from the retinal scan and/or for implementing additional functionality in the MSLO. For example, visible illumination may be used for improved contrast of retinal vasculature and ischemia (e.g., green or orange-yellow laser illumination) and/or to provide a visible fixation image to the patient whose retina is being scanned, as discussed further below. Accordingly, the laser illumination sub-assembly may include one or more visible lasers, such as a red laser 240 and/or blue laser 250 as illustrated in FIG. 8. As used herein the term “visible laser” is intended to refer to a laser configured to emit a beam having a wavelength (or wavelength range) in the visible part of the electromagnetic spectrum. The lasers 230, 240 and 250 may be any type of suitable laser source, such as laser diodes or fiber lasers for example. Focusing optics 260 may be used to focus and/or collimate the output beams from the lasers 230, 240 and 250. In some embodiments, configuration of the laser packaging and/or arrangement of the laser illumination sub-assembly within the housing of the MSLO may result in one or more of the lasers not being directly in line with the desired pointing direction of the illumination beams 210. Accordingly, a fold minor 270 may be used to redirect the laser beams from one or more the lasers. Beam splitters 280 may be used to allow different lasers to share the same optical pathways.

According to one embodiment, detection in the MSLO is performed by placing a detector, such as a photo-multiplier tube, avalanche photodiode, or charge-coupled device (CCD), in the same light path where the incident illumination beam 210 originates, thereby creating a reverse scan using the same scanning mirror 300. As illustrated in FIG. 1B, the beam splitter 120 may be used to allow the illumination beam 210 and return beam 510 to share a portion of the same optical path. As discussed above, in one example the illumination beam is approximately 1-2 mm in diameter; however, light scattered from the retina 118 back through a 3-5 mm diameter human pupil at the VSP may increase in diameter to nearly 10 mm at the RSP due to beam magnification in the return path from the front objective 400.

Therefore, the free aperture of the MEMS scanning minor 300 may be significantly smaller than the beam diameter of the return beam. For example, in one embodiment, the MEMS scanning minor 300 is approximately 2 mm in diameter. As a result, absent a mechanism to compensate for the difference in size between the return beam 510 and the scanning mirror 300, a large percentage of the scattered rays in the return beam 510 may not be reverse scanned off the scanning minor 300 (because they are not incident on the scanning minor) and therefore could be undetected. Accordingly, embodiments of the MSLO include mechanisms for increasing the detection sensitivity and capturing a large percentage of the scattered light.

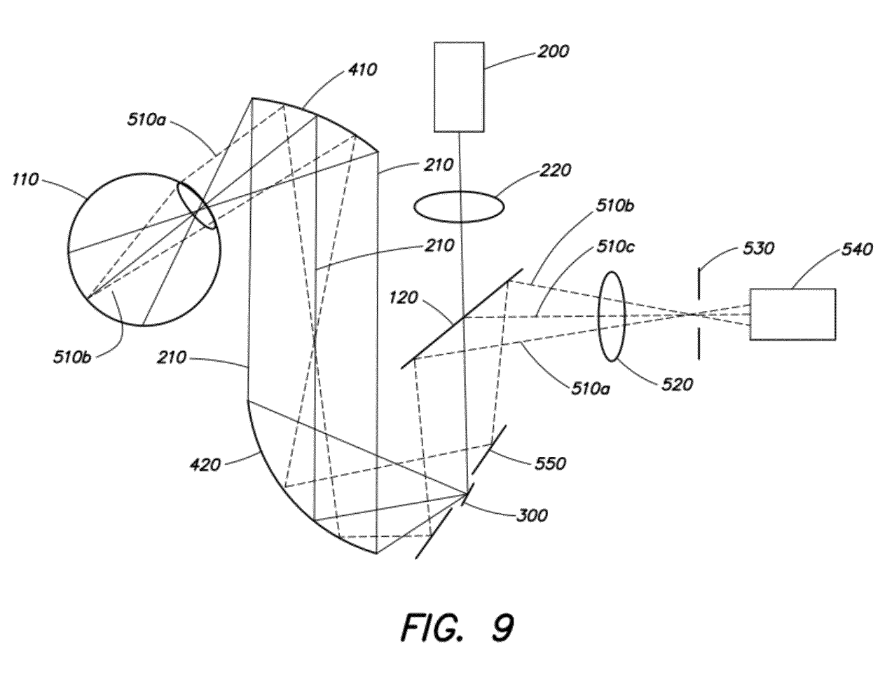

Referring to FIG. 9, in one embodiment a holed mirror 550 is placed immediately above the plane of the MEMS scanning minor 300 to capture more of the light returning from the retina 118 than is captured using traditional reverse scanning approaches. The holed mirror 550 is positioned with the hole placed over the MEMS scanning minor 300 to allow the illumination beam 210 to pass through to the eye 110. In FIG. 9, the solid lines 210 represent the illumination beam at three different points in time, corresponding to three different deflection positions of the scanning mirror 300. The holed minor 550 efficiently captures light reflected from retina 118 and directs the reflected light to the detector(s) 540. The dotted lines 510a and 510b represent the extreme rays of the reflected light from the retina 118 at a single point in time. The dotted line 510c represents the central ray of the reflected light, which is overlapped with the illumination beam 210 in the shared optical pathway when the scanning minor 300 is in its central or neutral deflection position (0 degrees).

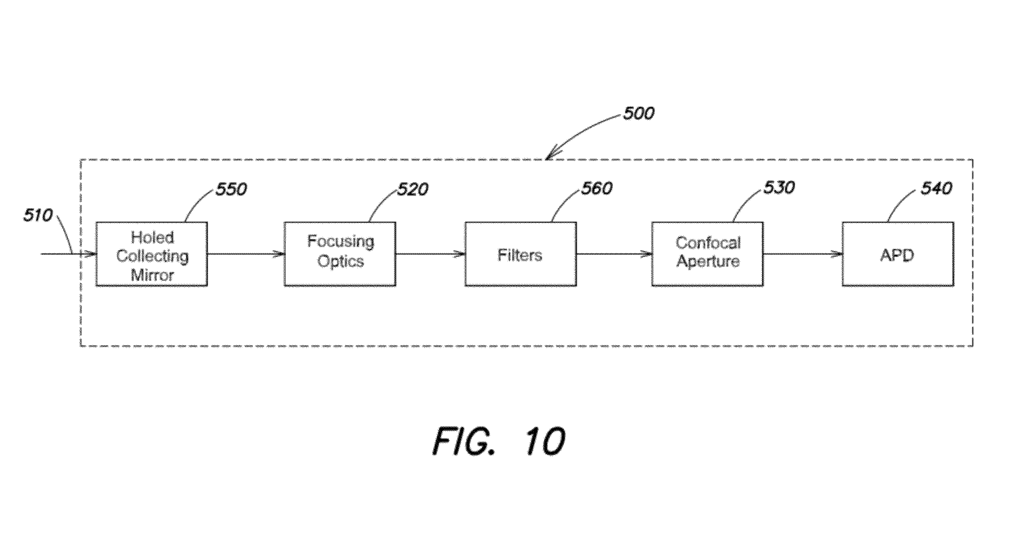

Referring to FIG. 10 there is illustrated a functional block diagram of one example of a detector sub-assembly 500 according to one embodiment. In the illustrated example, the detector sub-assembly 500 includes a holed mirror 550, as discussed above, that directs the return beams 510 to focusing optics 520. The focusing optics 520 focuses and direct the return beams 510 to the confocal aperture 530, as illustrated for example in FIG. 9. The confocal aperture 530 may be used to filter light reflected from tissue layers outside of focal plane. The detector sub-assembly may optionally include one or more color filters 560 that selectively pass the wavelengths of the return beam 510. As discussed above, the detector 540 may be any type of suitable photodetector including, for example, an avalanche photodiode, CCD or photo-multiplier tube. The output from the detector may be stored and/or provided to processor, either integrated with the MSLO or remote, for analysis.

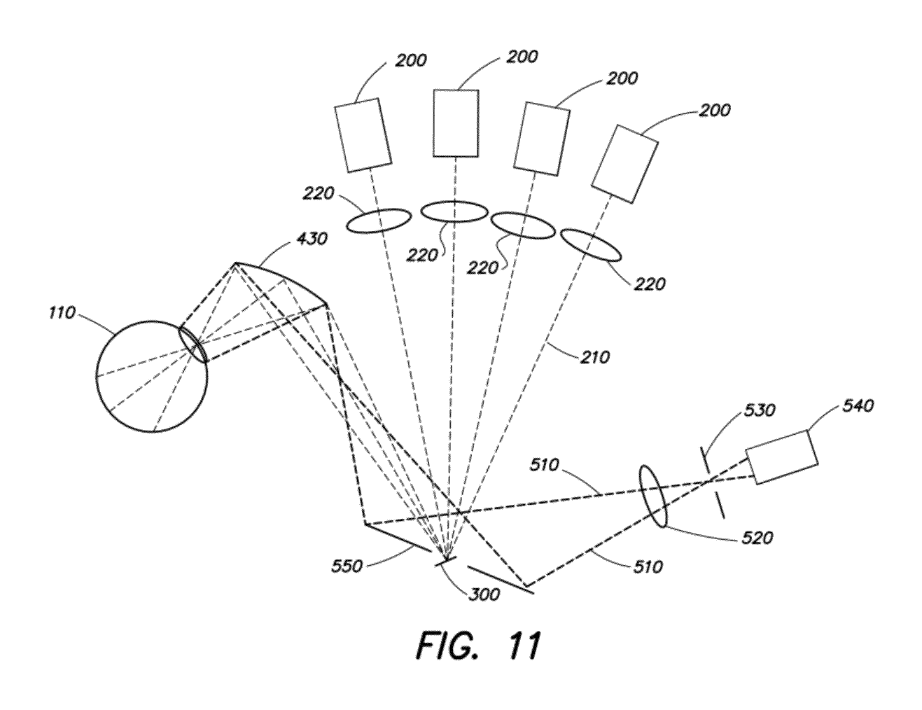

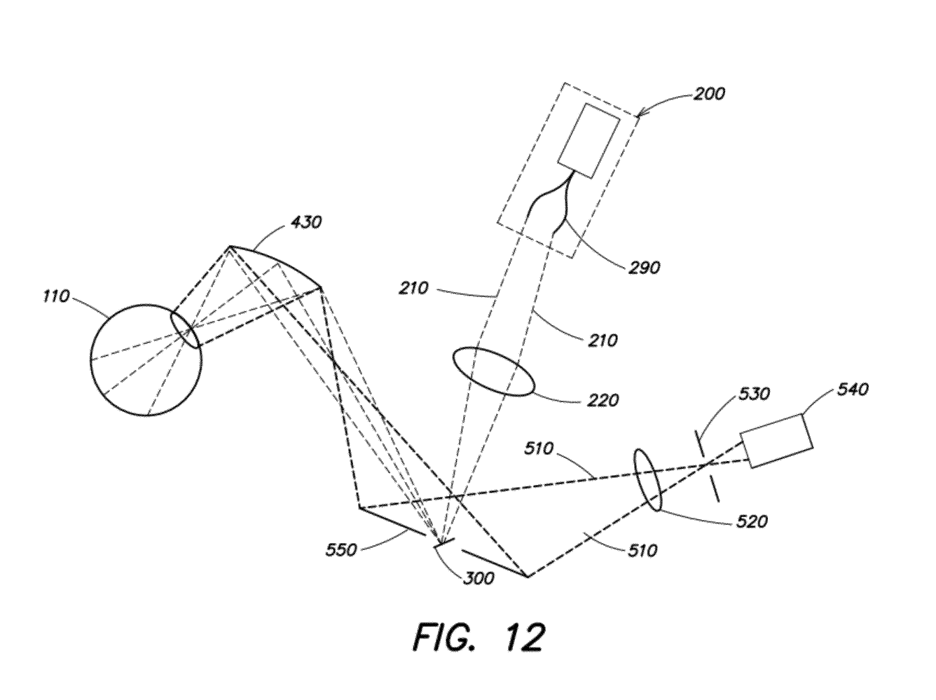

Numerous configurations of the MSLO including some or all of the features and components discussed above may be implemented. For example, as discussed above, embodiments of the MSLO may include a plurality of laser illumination sub-assemblies to generate illumination beams incident on the MEMS scanning minor 300 at different angles. FIG. 11 illustrates an example configuration of an MSLO including four laser illumination sub-assemblies 200, with a holed minor 550 positioned over the MEMS scanning mirror 300 as discussed above. In this example, the front objective includes an ellipsoid 430. In FIG. 11 the dotted lines 510 represent scattered rays from the retina 118 which are reflected by the holed minor 550 to the detector 540. As also discussed above, the laser illumination sub-assemblies 200 may be implemented using any of a variety of different types of lasers. The multiple illumination beams may also be generated by multiple individual laser illumination assemblies 200, as illustrated in FIG. 11, or using an array of lasers within one or more laser illumination sub-assemblies. For example, FIG. 12 illustrates an example of an MSLO in which a laser illumination sub-assembly 200 includes a fiber optic laser array configured to produce multiple illumination beams 210. It is to be appreciated that various laser illuminators may be used, not limited to the illustrated examples. In any of the examples discussed and/or illustrated herein, one type or configuration of laser illumination sub-assembly may be replaced with another. Similarly, in any example, one type of front objective 400 may be replaced with another. For example, a configuration using a parabolic front objective may be modified to use an ellipsoid front objective, and vice versa.

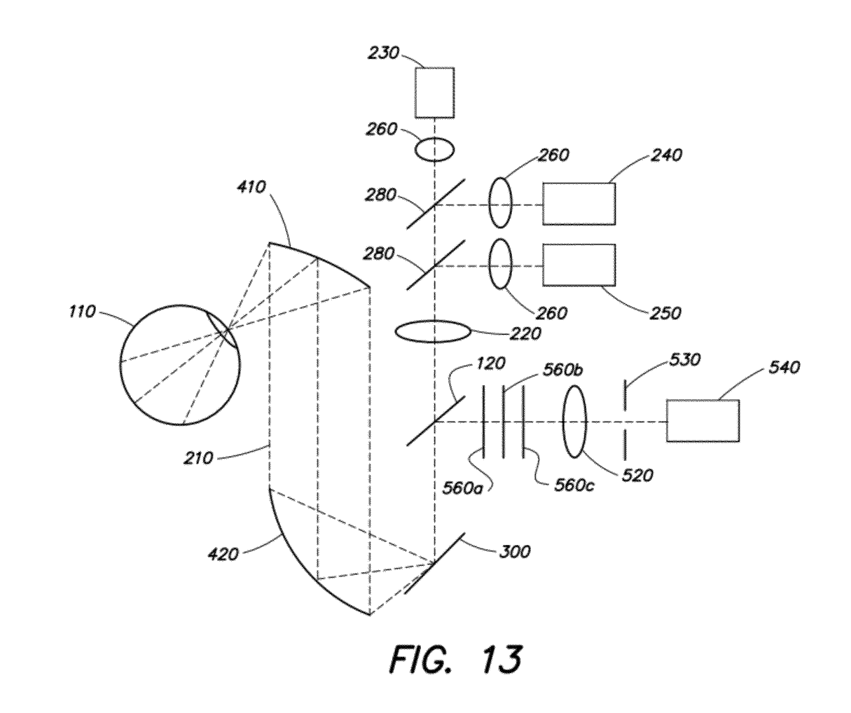

As discussed above, according to one embodiment, the laser illumination sub-assembly 200 may be configured to produce illumination beams of different wavelengths. For example, near-infrared may be used for imaging the retina and visible light may be used to present a fixation target to a human subject, as discussed further below. An example of a configuration of an MSLO using different illuminators 230, 240 and 250 configured to lase at different wavelengths is illustrated in FIG. 13. In this example, the detector sub-assembly includes filters 560a-c, each of which may be matched to the wavelength of a corresponding illuminator 230, 240, 250, respectively.

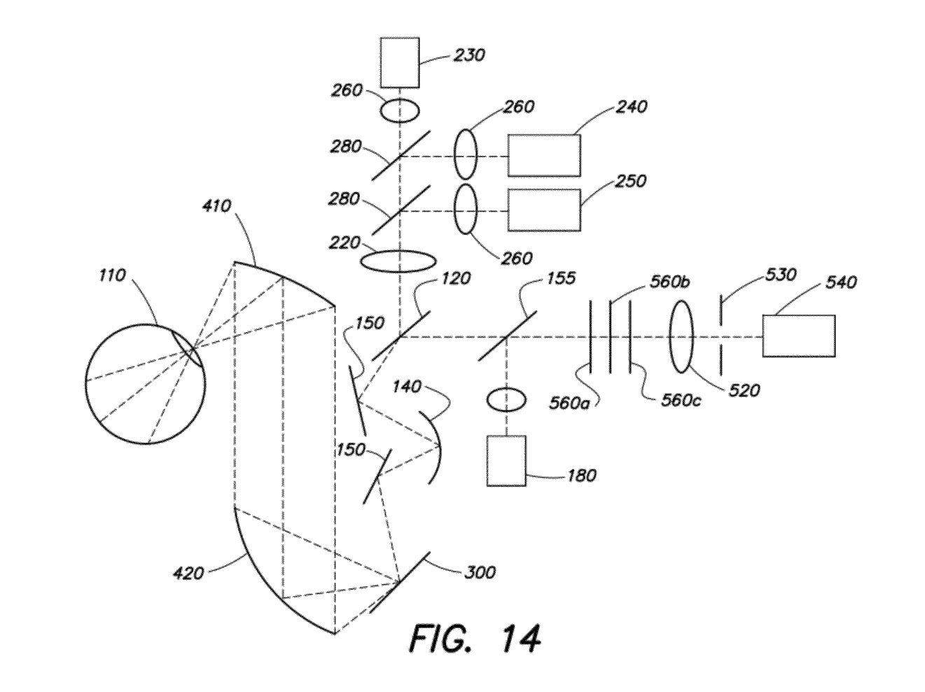

In another example, illustrated in FIG. 14, the MSLO includes adaptive optics 140. Fold minors 150 may be used to direct the illumination beams to and from the adaptive optics 140. The adaptive optics may be used to correct for human eye aberrations to allow for improved spatial resolution on the retina 118. In one example, a wavefront sensor 180 is configured to measure the change the wavefront of the optical beam(s) due to aberrations in the eye and is used in a feedback control loop to control the adaptive optics to compensate for the aberrations. A beam splitter 155 may be used to direct a portion of the optical beam(s) to the wavefront sensor 180. Some embodiments, particularly where obtaining best spatial resolution on the retina is important, may include adaptive optics; however, more compact, light-weight and low-power embodiments may be implemented without including adaptive optics since the adaptive optics may increase the size, weight and power requirements of the MSLO.

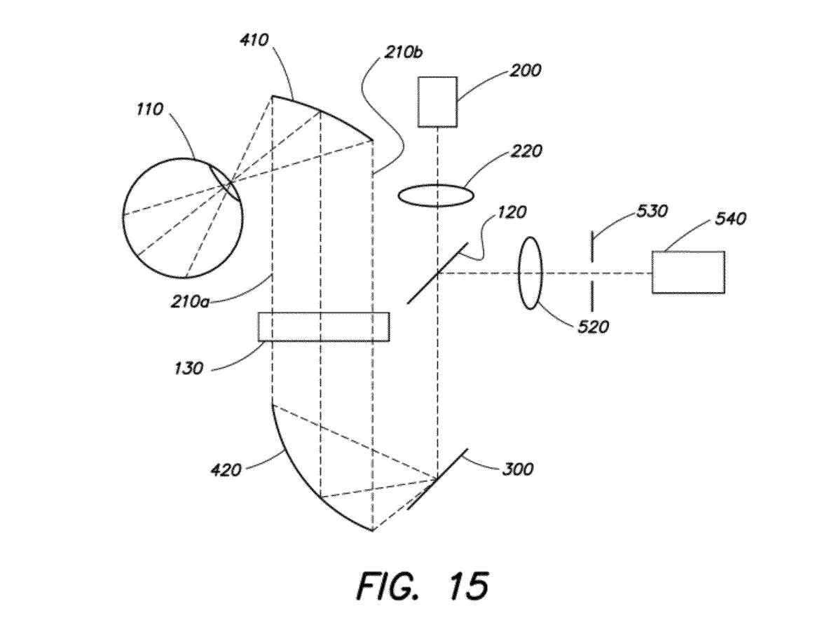

In another embodiment, to compensate for path length differences at different points in the scan, a variable optical delay block 130 may be inserted between the two paraboloids 410, 420 of the front objective, as illustrated in FIG. 15. The optical path length between extremes of the scan, represented in FIG. 15 by illumination beams 210a and 210b, may be significantly different. As a result, the focus of the illumination beam on the retina of the eye 110 may change over the scan. Accordingly, to compensate for this variation in path length and potential loss of focus, a variable optical delay block 130 may add varying amounts of delay to the various optical pathways, and thereby substantially equalize the optical path length over the scan.

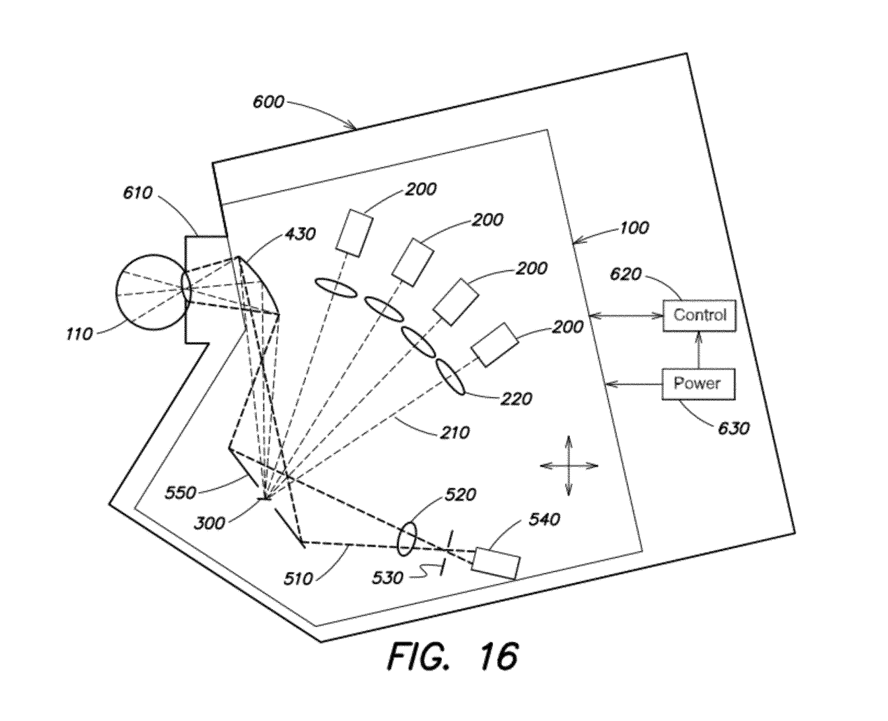

According to one embodiment, to enable automated, self-retinal imaging, the entire MSLO subsystem 100 described above is enclosed within a housing and electro-mechanically actuated to travel with six degrees of freedom so that the VSP can be moved around until the retina 118 of the human subject is in best focus. FIG. 16 illustrates an overhead view of one example of the MSLO device with the optical subsystem arranged within a housing/enclosure 600. In one embodiment, the housing 600 includes an eyepiece portion 610 configured such that the patient can hold the device to their eye 110 to perform a self-retinal scan. The MSLO device may also include a controller 620 and a power supply 630 located within the housing 600. In one example the power supply 630 includes a battery. The power supply may provide power to any active components in the MSLO, including the laser illumination sub-assembly 200 for example, as well as to the controller 620.

The controller 620 may be configured to control various components and aspects of operation of the MSLO 100 to perform scanning of the patient's eye 110. For example, in embodiments in which one or more laser illumination sub-assemblies 200 include the ability to generate illumination beams 210 at different wavelengths, the controller 620 may control the wavelength(s) of light used for the illumination and/or the order in which beams of different wavelengths are scanned. The controller 620 may further control any active components, such as adaptive optics 140, which may be included in the MSLO 100. The controller 620 may further control the processing, storage and/or transmission to a remote location of the output from the detector sub-assembly 500, as discussed further below. According to a variety of examples, the controller 620 includes a commercially available processor such as processors manufactured by Texas Instruments, Intel, AMD, Sun, IBM, Motorola, Freescale and ARM Holdings. However, the controller 620 may be any type of processor, field-programmable gate array, multiprocessor or controller, whether commercially available or specially manufactured.

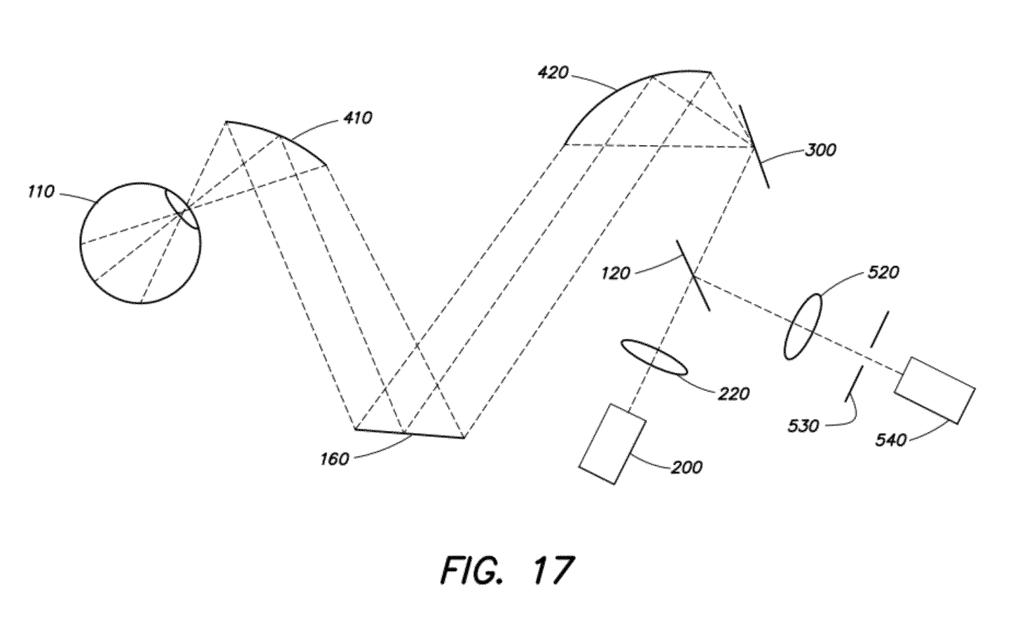

The MSLO 100 may have any of numerous configurations (examples of which are discussed above) within the housing 600. In some embodiments, the physical structure/configuration of the housing 600 and/or arrangement of the MSLO 100, controller 620 and power supply 630 within the housing may affect the layout of the components of the MSLO, and optionally the optical configuration selected for the MSLO. In one example in which the MSLO includes a double paraboloid front objective 400, one or more relay mirrors 160 (also referred to as fold mirrors) may be used to rotate the axis of the second paraboloid 420 relative to the first paraboloid 410, as illustrated in FIG. 17, should space, clearance and/or arrangement of the optical components within the MSLO housing make this desirable.

According to one embodiment, a two-dimensional scan of the retina 118 of a patient's eye 110 is performed by scanning the illumination beams 210 over the retina 118 in two dimensions. As discussed above, two or more illumination beams 210 in each dimension may be used to achieve a fast, high resolution, wide angle scan. The 2D MEMS scanning mirror 300 implements a “raster” scan by “tilting” over its range of angular motion 320 in both dimensions. In one example, the scanning minor 300 has a fast dimension and a slow dimension, as is the case in conventional television raster scanning. However, the scan need not be rectangular; instead the scanning minor 300 may be configured to implement a spiral or vector raster scan. In one example, the power supply 630 supplies a varying voltage to the 2D MEMS scanning mirror 300 to activate the minor to move over its range of angular motion (or selected portion thereof) to perform the scan. The scattered light from the retina 118 forms the return beams 510 which are collected by the detector sub-assembly 500. The detector 540 provides an output based on the detected return beams 510, and the output is processed to provide an image of retina. Image processing of the detector output may be performed, at least in part, by the controller 620. In one example, the controller 620 includes a storage device (not shown) for storing the detector output (raw or processed) to be provided to a remote user. For example, the controller may include a communications interface to transmit the detector output to a remote location for processing and/or analysis, or the storage may be removable from the housing 600 to allow the data stored thereon to be processed and/or analyzed on another machine. In one example, the storage includes non-transient computer-readable random access memory such as dynamic random access memory (DRAM), static memory (SRAM) or synchronous DRAM. However, the storage may include any device for storing data, such as non-volatile memory, with sufficient throughput and storage capacity to support the functions described herein.

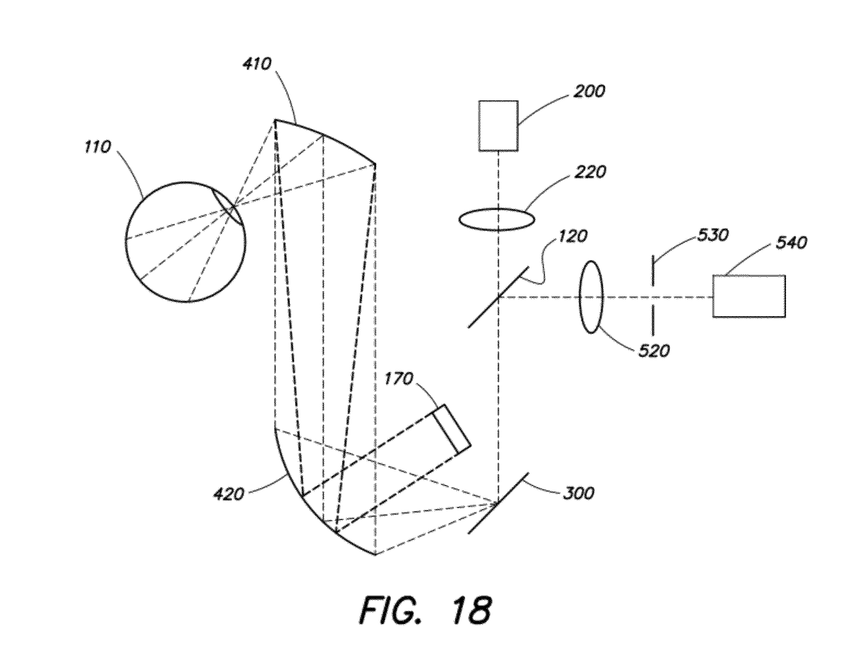

In one embodiment, in operation of the MSLO, the human subject peers through the eyepiece 610 and the MSLO continually scans illumination (for example, near-infrared, ˜780 nm), captures the response at the detector 540, and relies on an internal feedback loop to adjust the position of the VSP. As discussed above, in one embodiment, a fixation target is presented to the human subject to guide the subject's eye 110 to a desired location/angle to obtain images of certain areas of the retina 118. In one example, the fixation target is presented as an image formed by modulating a visible laser (e.g., ˜520 nm or ˜635 nm) at appropriate times in the raster scan. The image location may automatically adjust to guide the subject's eye to the best location for imaging, as discussed further below. Accordingly, in one embodiment, the MSLO includes a display screen 170, for example, an LCD screen, as illustrated in FIG. 18, positioned such that it is visible to the patient looking into the eyepiece 610. The fixation target may be displayed on the display screen 170 and guide the patient's viewing direction. A new retinal region may be imaged by adjusting the location of the fixation target and instructing the user to look at the new target location. Presentation of fixation targets with real-time feedback and optional audio instructions to the patient advantageously allows for fully automated, self-administered retinal imaging.

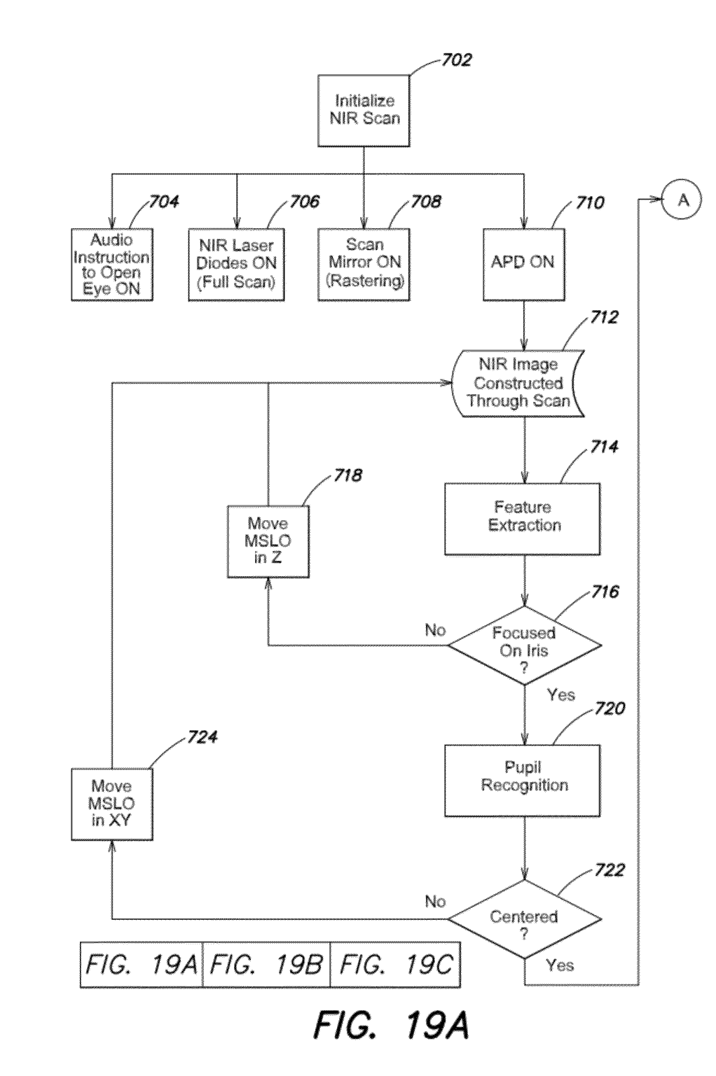

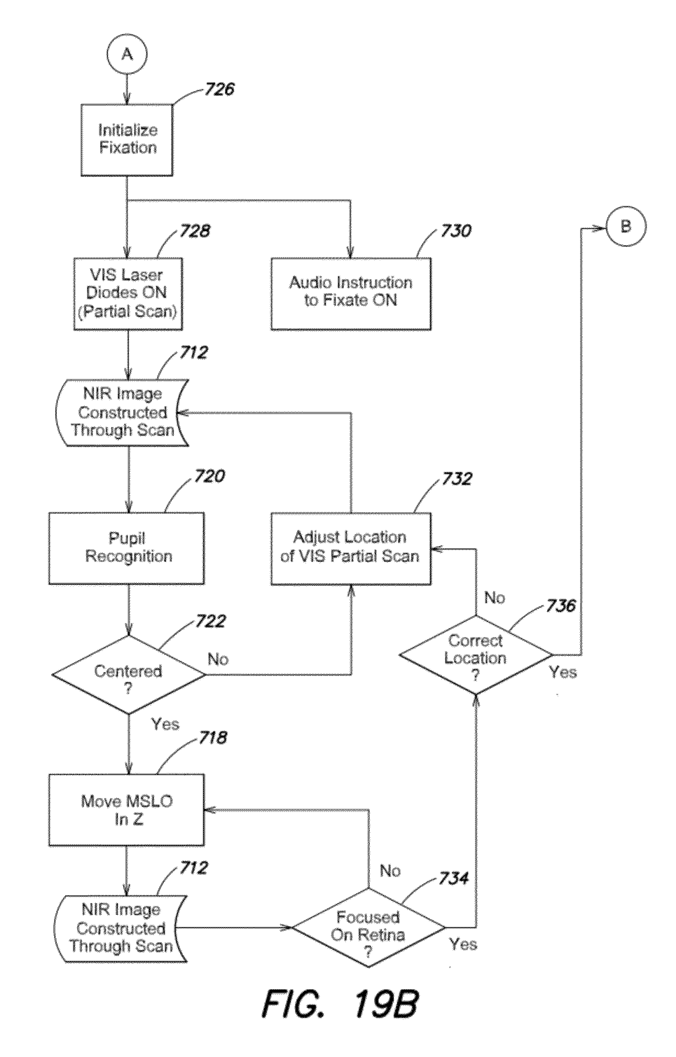

Referring to FIG. 19 there is illustrated a flow diagram of one example of a retinal imaging process according to one embodiment. To begin a scan, a first step 702 may include initializing the scan at a desired wavelength. For example, an imaging scan of the retina 118 may be performed using a near-infrared laser as discussed above. Step 702 may begin when a user turns on the MSLO device, for example. Initializing the imaging scan may include instructing the patient to look into the eye piece and open their eye 110 (step 704). This instruction may be audible (for example, the MSLO device may include a speaker (not shown) and the controller 620 may direct the speaker to audibly project the instruction) and/or visual, as discussed further below. Initializing the imaging scan may also include turning on the laser illumination sub-assembly 200 and activating the laser at the desired wavelength (step 706), turning on the scanning mirror 300 (step 708) and turning on the detector sub-assembly 500 (step 710). As discussed above, turning on the scanning minor 300 (step 708) may include controlling the power supply 630 to provide a varying voltage to the 2D MEMS scanning minor to actuate the mirror to continuously move through its range of angular deflection in each dimension. As the 2D MEMS scanning mirror moves, the illumination beam(s) 210 are moved across the retina of the eye 110 to obtain an image of the retina (step 712), as discussed above. In the example where the illumination laser is a near-infrared laser, the image obtained in step 712 is a near-infrared image.

According to one embodiment, the first scan after initialization (step 702) is used to determine whether the patient's eye 110 is oriented correctly for imaging a desired region of the retina and whether the eye is in focus. Accordingly, the controller 620 may process the image obtained in step 712, for example, by performing feature extraction processing (step 714) to locate specific points in the image, for example the iris, the pupil and/or portions of the retina. Following the feature extraction processing (step 714), the controller may determine whether or not the illumination beam(s) are focused on the iris of the eye 110 (step 716). If the iris is not in focus, the controller may move the MSLO 100 (or certain optical components thereof) in the z-direction (step 718). In one embodiment, the MSLO 100 may be mounted on movable linear stages within the housing 600 to allow movement of the MSLO (or at least certain optical components thereof) in the x-, y- and z-axes (forward and back, left and right, up and down). After moving the MSLO in step 718, a new image may be obtained in step 712 and processed in step 714 to determine whether or not the iris is now in focus (step 716). This process may be repeated until the iris is correctly focused in the image. The controller 620 may then process the image to locate the pupil of the eye 110 in the image (step 720) and determine whether or not the pupil is centered (step 722). If the pupil is not centered, the controller 620 may control the movable linear stages discussed above to move the MSLO 100 along the x- and/or y-axes (step 724) until the pupil is centered in the image.

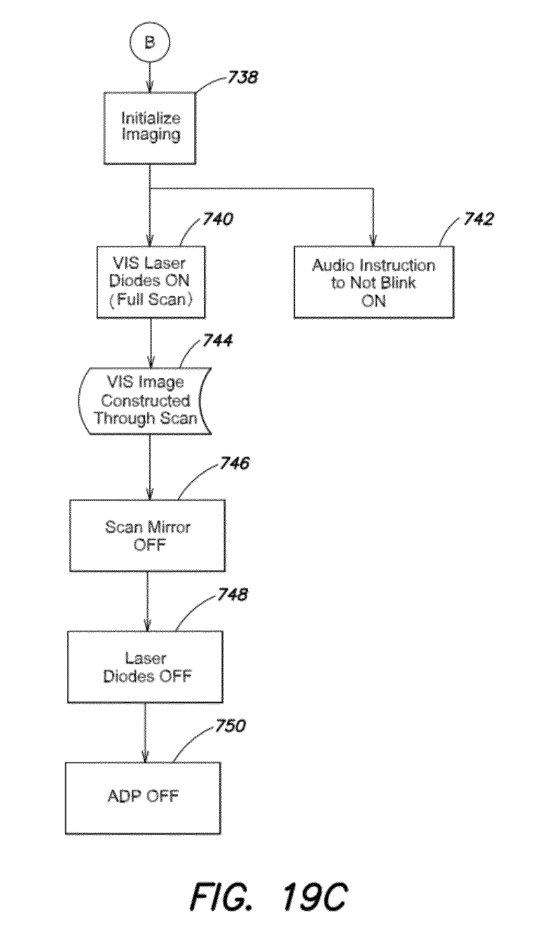

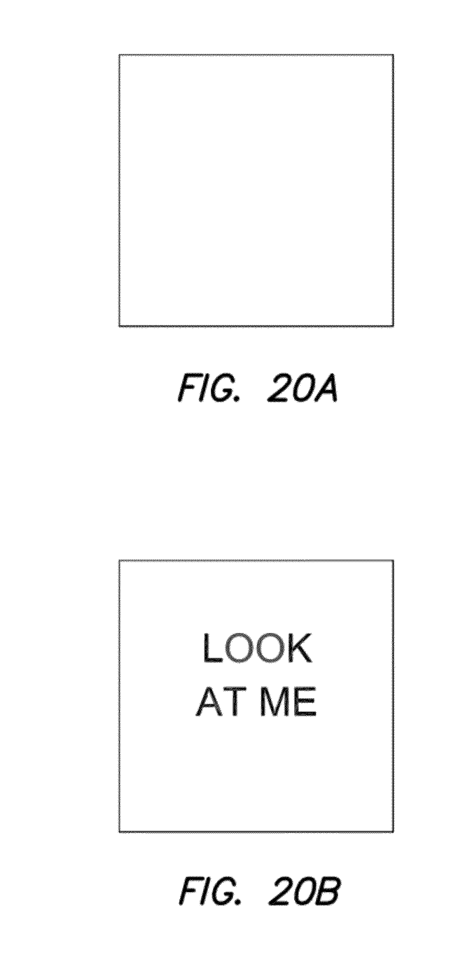

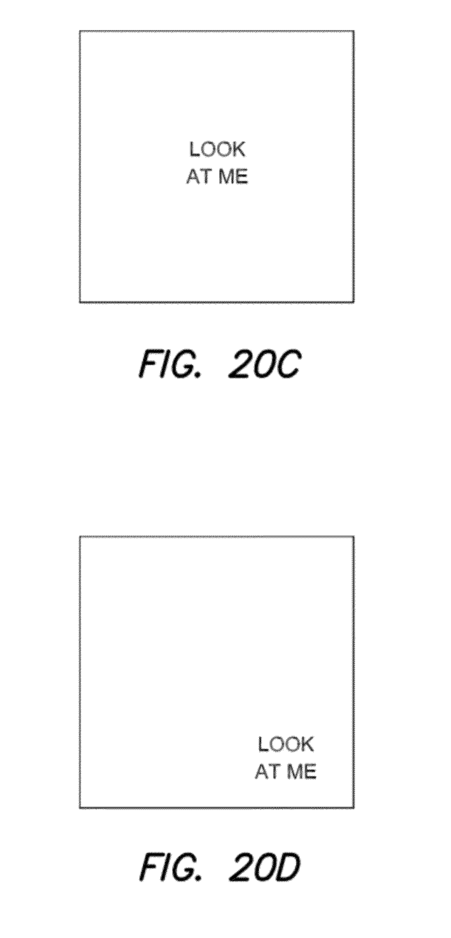

After initial set-up has been completed, the system may be configured to perform one or more scans of desired regions of the retina, using presentation of fixation targets with real-time feedback to enable fully automated, self-administered retinal imaging. As discussed above, in one example, the retinal image is obtained using infrared illumination. At the same time as the infrared scan is being performed, visible illumination is modulated appropriately to draw a fixation target at appropriate locations so that the human eye is oriented correctly to image the desired region of the retina. Thus, referring to FIG. 19, in one embodiment, step 726 includes initializing the fixation process, including activating one or more visible lasers to project the fixation target (step 728) and projecting an audible instruction to the patient to look at the fixation target (step 730). During the initial infrared set-up scanning discussed above, the fixation display 170 may be blank, as illustrated in FIG. 20A. When the fixation process is activated, the fixation target is displayed on the screen 170, for example, as illustrated in FIG. 20B.

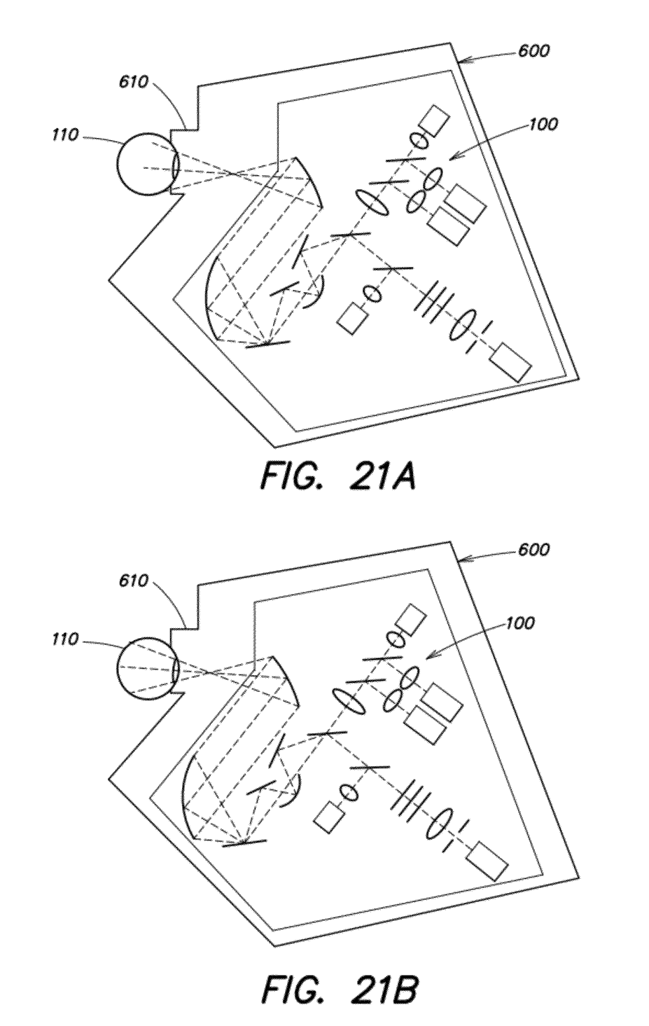

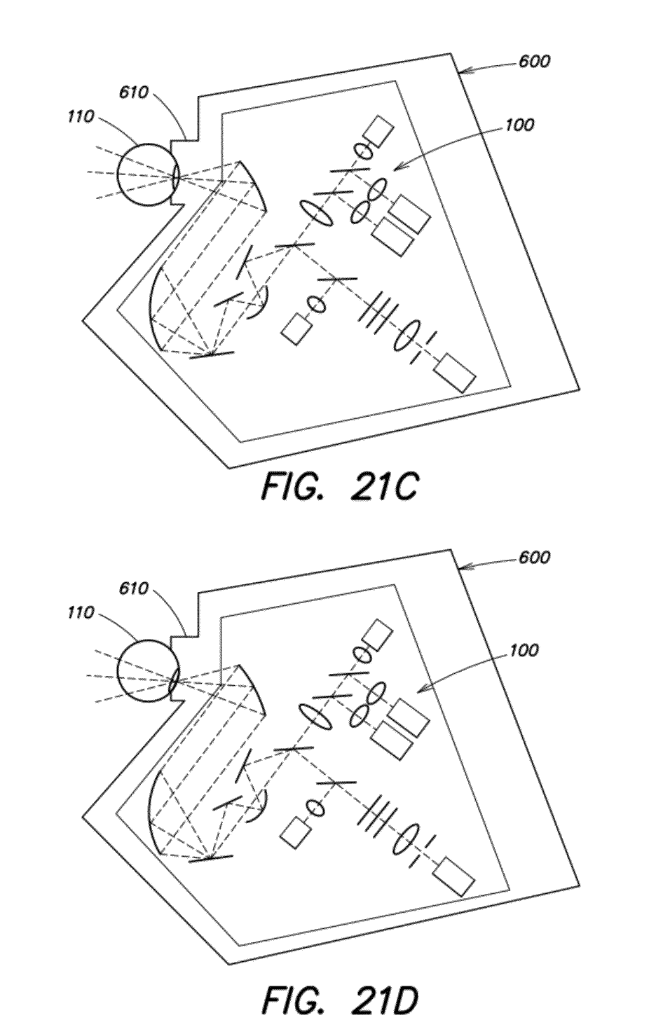

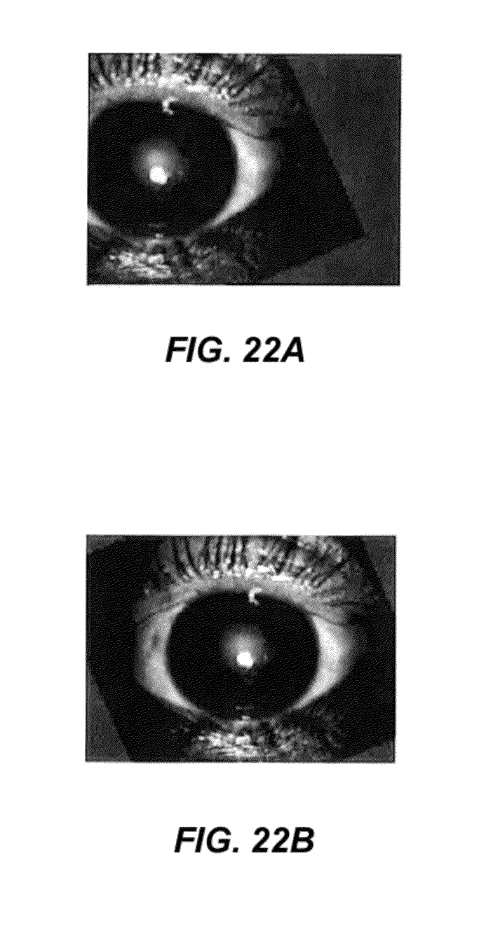

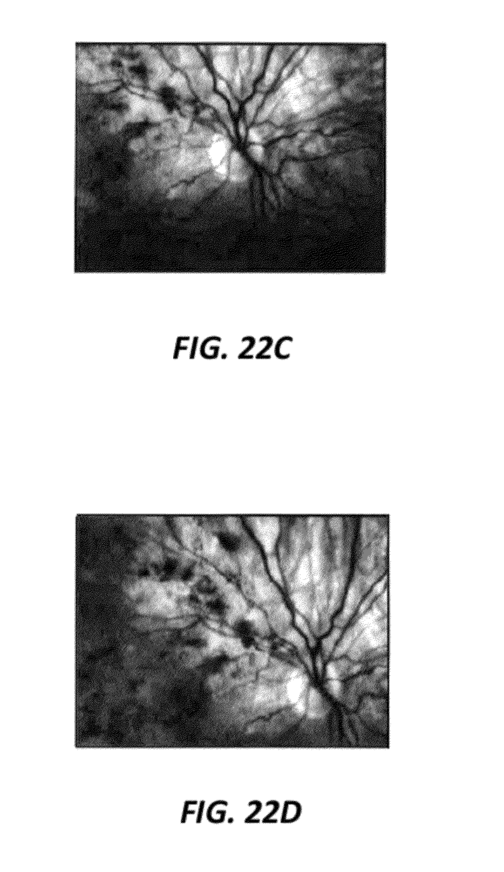

As illustrated in FIG. 21A, initially when the patient looks into the eyepiece 610, the eye 110 may not be oriented correctly, and accordingly the image obtained of the eye may be off-center, as illustrated in FIG. 22A. In one embodiment, the system is configured to obtain an image of the eye 110, e.g., using infrared illumination as discussed above, (step 712), recognize the pupil in the image (step 720) and determine whether the pupil is centered (step 722). If the pupil is not centered (as in FIG. 22A), the controller may control the system to adjust the location of the fixation target (step 732) until the eye is oriented (FIG. 21B) such that the pupil is centered in the image, as illustrated in FIG. 22B. The controller may then analyze the image to determine whether or not the retina is in focus (step 734) and move the MSLO or an internal focusing optic in the z-direction (step 718; FIG. 21C) until the retina is in focus, as illustrated in FIG. 22C. In one example, the fixation target is reduced in size as the MSLO nears the eye 110, as illustrated in FIG. 20C. In some instances, the retina may be in focus, but the area of interest may not be in view. Accordingly, the controller may determine whether or not the correct area of the retina is visible (step 736) and if not, move the fixation target to induce eye movement, as shown in FIG. 20D. The MSLO may move laterally to compensate for the eye 110 tracking the fixation target, as illustrated in FIG. 21D.

According to one embodiment, once the set-up has been completed and the correct area of the retina is in focus, the MSLO may be initialized (step 738) to perform one or more scans to obtain image(s) of the retina. These scans may use infrared and/or visual illumination and accordingly, the lasers to be used may be turned on (if not already on) and configured to perform a full scan (step 740). In one example, the system may project an audio instruction to the patient to not blink during the scan (step 742). The images are obtained by scanning the illumination beams across the retina using the 2D MEMS scanning mirror, as discussed above (step 744). The image(s) of interest may be stored for manual or automated analysis. An example of an image is illustrated in FIG. 22D. Various steps of the process may be repeated to obtain images of different areas of the retina and/or at different wavelengths to provide different information about the patient's retina. When all scans are complete, the system may be shut down, including powering off the scanning minor (step 746), the laser illumination sub-assemblies (step 748) and the detector sub-assembly (step 750).

Thus, aspects and embodiments provide a compact wide-field scanning laser ophthalmoscope using a 2D MEMS micromirror for fast scanning with few moving parts and robust portability in a light-weight package. As discussed above, embodiments of the MSLO may use multiple light paths/angles (e.g., multiple laser beams from different angles) incident on the scanning minor to magnify the scan angle (and increase the scanned field of view) without demagnifying the beam diameter. In addition, a conic objective (e.g., a double paraboloid or ellipsoid) is used to translate the scan over a wide field of view in the human eye. As discussed above, embodiments of MSLO may enable wide-field scanning of a 1 mm diameter beam about a virtual scanning point into the eye, thereby achieving approximately 10 μm lateral resolution. In some embodiments, a holed minor is placed in front of scanning element so that all common light path optics are reflective, and more light from the retina is returned to the detector. In addition, adjustable fixation targets may be presented to human subjects with real-time feedback to enable fully automated (including auto-alignment, auto-focus, and auto-capture/image acquisition), self-administered retinal imaging, as discussed above. Embodiments of the MSLO may make possible self-administered retinal imaging in any location, allowing for earlier diagnosis of eye disease, which will reduce blindness and improve worldwide health.

Having described above several aspects of at least one embodiment, it is to be appreciated various alterations, modifications, and improvements will readily occur to those skilled in the art given the benefit of this disclosure. For example, any of the illustrated examples may in implementation include additional components; for example, although a filter 560 or holed minor 550 is not illustrated in certain examples, the detector sub-assemblies in these and other examples may include one or more filters 560 and/or the holed minor 550. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

What is claimed is:

1. A MEMS-based scanning laser ophthalmoscope comprising:
a laser illumination sub-assembly configured to generate a laser illumination beam;
a two-dimensional MEMS scanning mirror configured to receive the laser illumination beam and to produce a two-dimensional area of illumination;
an optical system optically coupled to the MEMS scanning minor and configured to direct the two-dimensional area of illumination from the scanning mirror into an eye to illuminate a retina of the eye; and
a detector sub-assembly optically coupled to the optical system and the MEMS scanning mirror and configured to intercept optical radiation reflected from the eye to generate an image of the retina.

2. The MEMS-based scanning laser ophthalmoscope of claim 1, wherein the detector sub-assembly includes a photodetector and a holed mirror, the holed minor being positioned over the two-dimensional MEMS scanning minor and configured and arranged to allow the laser illumination beam to pass through an opening in the holed mirror to the optical system, and to direct the optical radiation reflected from the eye to the photodetector.

3. The MEMS-based scanning laser ophthalmoscope of claim 1, wherein the detector sub-assembly includes a photodetector, the photodetector comprising one of an avalanche photodiode, a charge coupled device, and a photo-multiplier tube.

4. The MEMS-based scanning laser ophthalmoscope of claim 3, wherein the detector sub-assembly further includes a focusing optic configured to focus the optical radiation to the photodetector.

5. The MEMS-based scanning laser ophthalmoscope of claim 4, wherein the detector sub-assembly further includes a confocal aperture optically coupled between the focusing optic and the photodetector.

6. The MEMS-based scanning laser ophthalmoscope of claim 1, wherein the laser illumination beam includes at least two first illumination beams spaced apart from one another by a first angle of separation in a first dimension, and at least two second illumination beams spaced apart from another by a second angle of separation in a second orthogonal dimension.

7. The MEMS-based scanning laser ophthalmoscope of claim 1, wherein the optical system includes a conic front objective having two foci, and wherein the two-dimensional MEMS scanning mirror is located at a first focus of the conic front objective and the MEMS-based scanning laser ophthalmoscope is configured to accommodate a pupil of the eye at a second focus of the conic front objective.

8. The MEMS-based scanning laser ophthalmoscope of claim 1, wherein the laser illumination sub-assembly includes a near-infrared laser source.

9. The MEMS-based scanning laser ophthalmoscope of claim 1, wherein the laser illumination sub-assembly includes at least one visible laser source.

10. The MEMS-based scanning laser ophthalmoscope of claim 1, further comprising a battery configured to provide power to the two-dimensional MEMS scanning mirror and to the laser illumination sub-assembly.

11. The MEMS-based scanning laser ophthalmoscope of claim 1, further comprising:
a display screen optically coupled to the optical system; and
a controller configured to control the laser illumination sub-assembly to display a fixation target on the display screen.

12. The MEMS-based scanning laser ophthalmoscope of claim 11, wherein the controller is further configured to adjust a display location of the fixation target on the display screen to guide an orientation of the eye so as to obtain an image of a selected region of the retina.

13. The MEMS-based scanning laser ophthalmoscope of claim 12, wherein the laser illumination sub-assembly includes a visible laser source configured to provide visible laser illumination, and wherein the visual laser illumination is modulated to produce the fixation target displayed on the display screen.

14. A method of imaging a retina of an eye with a scanning laser ophthalmoscope, the method comprising:
generating laser illumination;
scanning the laser illumination about a scan point at the eye using a two-dimensional MEMS scanning minor to produce a two-dimensional area of illumination that illuminates the retina of the eye;
intercepting optical radiation reflected from the eye;
acquiring a first image of the eye from the optical radiation;
analyzing the first image of the eye to identify features in the first image; and
based on the features, automatically adjusting at least one of an alignment and a focus of optical components of the scanning laser ophthalmoscope to obtained a focused image of a selected region of the retina of the eye.

15. The method of claim 14, wherein analyzing the image of the eye includes determining whether a pupil of the eye is centered with respect to the laser illumination; and further comprising:
laterally moving optical components of the scanning laser ophthalmoscope and acquiring subsequent images of the eye until the pupil is centered in one of the subsequent images.

16. The method of claim 14, wherein analyzing the image of the eye includes determining whether the retina of the eye is in focus; and further comprising:
moving the optical components of the scanning laser ophthalmoscope and acquiring additional images of the eye until the retina is in focus in one of the additional images.

17. The method of claim 14, further comprising:
displaying a fixation target to guide an orientation of the eye so as to obtain an image the selected region of the retina of the eye.

18. The method of claim 17, further comprising:
adjusting a display location of the fixation target to guide the orientation of the eye so as to obtain an image of another selected region of the retina of the eye.

19. The method of claim 14, wherein generating the laser illumination includes generating in each of two orthogonal dimensions at least two illumination beams separated from one another by a predetermined angle of separation.

20. A method of imaging a retina of an eye with a scanning laser ophthalmoscope, the method comprising:
generating a laser illumination beam;
scanning the laser illumination beam about a scan point at the eye using a two-dimensional MEMS scanning mirror to produce a two-dimensional area of illumination that illuminates the retina of the eye;
intercepting optical radiation reflected from the eye; and
producing an image of retina from the optical radiation.

21. The method of claim 20, wherein generating the laser illumination beam includes generating at least one of an infra-red illumination beam and a visible illumination beam.

22. The method of claim 21, wherein intercepting the optical radiation reflected from the eye includes detecting the optical radiation using one of a photo-multiplier tube, a charge coupled device, and an avalanche photodiode.

23. The method of claim 20, further comprising:
displaying a fixation target on a display screen; and
adjusting a display location of the fixation target on the display screen to guide an orientation of the eye so as to obtain an image of a selected region of the retina.
